(19) United States
(12) Patent Application Publication
(10) Pub. No.: US 2025/0176879 A1
(43) Pub. Date: Jun. 5, 2025

(54) ANALYTE SENSOR START UP

(71) Applicant: Dexcom, Inc., San Diego, CA (US)

(72) Inventors: Liang Wang, San Deigo, CA (US); Hossein Mohammadiarani, San Diego, CA (US); Neda Ehtiati, San Diego, CA (US); Ghazaleh R Esmaili, San Diego, CA (US); Jonathan M Hughes, Carlsbad, CA (US); Arun Panch Santhanam, San Diego, CA (US); Wenjie Lan, Westford, MA (US); Daiting Rong, San Diego, CA (US)

(21) Appl. No.: 18/969,826

(22) Filed: Dec. 5, 2024

Related U.S. Application Data
(60) Provisional application No. 63/606,497, filed on Dec. 5, 2023.

(51) Int. Cl. A61B 5/1495; A61B 5/00; A61B 5/145

(52) U.S. Cl. CPC A61B 5/1495; A61B 5/14503; A61B 5/14532; A61B 5/6849; A61B 2560/0223

(57) ABSTRACT

Various examples are directed to systems and methods for operating an analyte sensor system. A sensor electronics of the analyte sensor system may access an indication that an analyte sensor has been inserted into a host. The sensor electronics may detect a property of a sensor signal generated by the analyte sensor after being inserted into the host. The sensor electronics may determine a first sensor sensitivity based at least in part on the property of the sensor signal. The sensor electronics may also determine a first analyte concentration using the sensor signal and the first sensor sensitivity.

### CLAIM OF PRIORITY

This application claims priority to U.S. Provisional Application Ser. No. 63/606,497, filed Dec. 5, 2023, which is hereby incorporated by reference herein in its entirety.

### TECHNICAL FIELD

The present development relates generally to medical devices such as analyte sensors, and more particularly, but not by way of limitation, to systems, devices, and methods for starting up an analyte sensor.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels. A glucose sensor can provide an estimated glucose concentration level, which can be used as guidance by a patient or caregiver.

Diabetes conditions are sometimes referred to as “Type 1” and “Type 2.” A Type 1 diabetes patient is typically able to use insulin when it is present, but the body is unable to produce sufficient amounts of insulin because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Blood sugar concentration levels may be monitored with an analyte sensor, such as a continuous glucose monitor. A continuous glucose monitor is used by a host (e.g., patient) to provide information, such as an estimated blood glucose value or a trend of estimated blood glucose levels.

### SUMMARY

This present application discloses, among other things, systems, devices, and methods related to analyte sensors, including, for example, deploy testing in analyte sensors.

Example 1 is an analyte sensor system for in vivo use, the analyte sensor system comprising: an analyte sensor; and sensor electronics in electrical communication with the analyte sensor, the sensor electronics being configured to perform operations comprising: accessing an indication that the analyte sensor has been inserted into a host; detecting a property of a sensor signal generated by the analyte sensor after being inserted into the host; determining a first sensor sensitivity based at least in part on the property of the sensor signal; and determining a first analyte concentration using the sensor signal and the first sensor sensitivity.

In Example 2, the subject matter of Example 1 optionally includes the operations further comprising: determining that a break-in for the analyte sensor has concluded; and responsive to determining that the break-in has concluded, determining a second analyte concentration using the sensor signal and a second sensor sensitivity different than the first sensor sensitivity.

In Example 3, the subject matter of Example 2 optionally includes the determining that the break-in for the analyte sensor has concluded comprising determining that a break-in time period has elapsed since the analyte sensor was inserted into the host.

In Example 4, the subject matter of Example 3 optionally includes minutes.

In Example 5, the subject matter of any one or more of Examples 2-4 optionally includes the determining that the break-in for the analyte sensor has concluded comprising determining that a linearity of the sensor signal has reached a linearity threshold.

In Example 6, the subject matter of any one or more of Examples 1-5 optionally includes the detecting of the property of the sensor signal comprising: detecting a peak in the sensor signal; and determining a time-to-peak, the time-to-peak describing a time between when the analyte sensor was inserted into the host and the peak in the sensor signal.

In Example 7, the subject matter of any one or more of Examples 1-6 optionally includes the operations further comprising: after accessing the indication that the analyte sensor has been inserted into the host, sampling the sensor signal at a first sample rate; and after detecting the property of the sensor signal, sampling the sensor signal at a second sample rate, the second sample rate being less than the first sample rate.

In Example 8, the subject matter of Example 7 optionally includes seconds.

In Example 9, the subject matter of any one or more of Examples 1-8 optionally includes the first sensor sensitivity being based on at least one of a time between insertion of the analyte sensor and a rise in the sensor signal or a slope of the sensor signal after insertion.

In Example 10, the subject matter of any one or more of Examples 1-9 optionally includes the first sensor sensitivity being based at least in part on a first term based on a calibration of the analyte sensor and a second term based on the property of the sensor signal.

In Example 11, the subject matter of Example 10 optionally includes the calibration of the analyte sensor being a factory calibration performed during a manufacturing process of the analyte sensor.

In Example 12, the subject matter of any one or more of Examples 10-11 optionally includes the first sensor sensitivity being based on an additive combination of the first term and the second term.

In Example 13, the subject matter of any one or more of Examples 10-12 optionally includes the second term also being based on the calibration of the analyte sensor.

In Example 14, the subject matter of any one or more of Examples 1-13 optionally includes the first sensor sensitivity being based at least in part on a first term based on a calibration of the analyte sensor, a second term based on the property of the sensor signal, and a third term based on the calibration of the analyte sensor and the property of the sensor signal.

In Example 15, the subject matter of Example 14 optionally includes the first sensor sensitivity being based on an additive combination of the first term, the second term, and the third term.

Example 16 is a method of operating an analyte sensor system, the method comprising: accessing, by sensor electronics of the analyte sensor system, an indication that an analyte sensor has been inserted into a host; detecting, by the sensor electronics, a property of a sensor signal generated by the analyte sensor after being inserted into the host; determining, by the sensor electronics, a first sensor sensitivity based at least in part on the property of the sensor signal; and determining, by the sensor electronics, a first analyte concentration using the sensor signal and the first sensor sensitivity.

In Example 17, the subject matter of Example 16 optionally includes determining, by the sensor electronics, that a break-in for the analyte sensor has concluded; and responsive to determining that the break-in has concluded, determining, by the sensor electronics, a second analyte concentration using the sensor signal and a second sensor sensitivity different than the first sensor sensitivity.

In Example 18, the subject matter of Example 17 optionally includes the determining that the break-in for the analyte sensor has concluded comprising determining that a break-in time period has elapsed since the analyte sensor was inserted into the host.

In Example 19, the subject matter of Example 18 optionally includes minutes.

In Example 20, the subject matter of Example 19 optionally includes the determining that the break-in for the analyte sensor has concluded comprising determining that a linearity of the sensor signal has reached a linearity threshold.

In Example 21, the subject matter of any one or more of Examples 16-20 optionally includes the detecting of the property of the sensor signal comprising: detecting a peak in the sensor signal; and determining a time-to-peak, the time-to-peak describing a time between when the analyte sensor was inserted into the host and the peak in the sensor signal.

In Example 22, the subject matter of any one or more of Examples 16-21 optionally includes after accessing the indication that the analyte sensor has been inserted into the host, sampling the sensor signal at a first sample rate; and after detecting the property of the sensor signal, sampling the sensor signal at a second sample rate, the second sample rate being less than the first sample rate.

In Example 23, the subject matter of Example 22 optionally includes seconds.

In Example 24, the subject matter of any one or more of Examples 16-23 optionally includes the first sensor sensitivity being based on at least one of a time between insertion of the analyte sensor and a rise in the sensor signal or a slope of the sensor signal after insertion.

In Example 25, the subject matter of any one or more of Examples 16-24 optionally includes the first sensor sensitivity being based at least in part on a first term based on a calibration of the analyte sensor and a second term based on the property of the sensor signal.

In Example 26, the subject matter of Example 25 optionally includes the calibration of the analyte sensor being a factory calibration performed during a manufacturing process of the analyte sensor.

In Example 27, the subject matter of any one or more of Examples 25-26 optionally includes the first sensor sensitivity being based on an additive combination of the first term and the second term.

In Example 28, the subject matter of any one or more of Examples 25-27 optionally includes the second term also being based on the calibration of the analyte sensor.

In Example 29, the subject matter of any one or more of Examples 16-28 optionally includes the first sensor sensitivity being based at least in part on a first term based on a calibration of the analyte sensor, a second term based on the property of the sensor signal, and a third term based on the calibration of the analyte sensor and the property of the sensor signal.

In Example 30, the subject matter of Example 29 optionally includes the first sensor sensitivity being based on an additive combination of the first term, the second term, and the third term.

Example 31 is a non-transitory machine-readable medium comprising instructions thereon that, when executed by at least one processor, cause the processor to perform operations comprising: accessing an indication that an analyte sensor has been inserted into a host; detecting a property of a sensor signal generated by the analyte sensor after being inserted into the host; determining a first sensor sensitivity based at least in part on the property of the sensor signal; and determining a first analyte concentration using the sensor signal and the first sensor sensitivity.

In Example 32, the subject matter of Example 31 optionally includes determining that a break-in for the analyte sensor has concluded; and responsive to determining that the break-in has concluded, determining and a second analyte concentration using the sensor signal and a second sensor sensitivity different than the first sensor sensitivity.

In Example 33, the subject matter of Example 32 optionally includes the determining that the break-in for the analyte sensor has concluded comprising determining that a break-in time period has elapsed since the analyte sensor was inserted into the host.

In Example 34, the subject matter of Example 33 optionally includes minutes.

In Example 35, the subject matter of any one or more of Examples 32-34 optionally includes the determining that the break-in for the analyte sensor has concluded comprising determining that a linearity of the sensor signal has reached a linearity threshold.

In Example 36, the subject matter of any one or more of Examples 31-35 optionally includes the detecting of the property of the sensor signal comprising: detecting a peak in the sensor signal; and determining a time-to-peak, the time-to-peak describing a time between when the analyte sensor was inserted into the host and the peak in the sensor signal.

In Example 37, the subject matter of any one or more of Examples 31-36 optionally includes after accessing the indication that the analyte sensor has been inserted into the host, sampling the sensor signal at a first sample rate; and after detecting the property of the sensor signal, sampling the sensor signal at a second sample rate, the second sample rate being less than the first sample rate.

In Example 38, the subject matter of Example 37 optionally includes seconds.

In Example 39, the subject matter of any one or more of Examples 31-38 optionally includes the first sensor sensitivity being based on at least one of a time between insertion of the analyte sensor and a rise in the sensor signal or a slope of the sensor signal after insertion.

In Example 40, the subject matter of any one or more of Examples 31-39 optionally includes the first sensor sensitivity being based at least in part on a first term based on a calibration of the analyte sensor and a second term based on the property of the sensor signal.

In Example 41, the subject matter of Example 40 optionally includes the calibration of the analyte sensor being a factory calibration performed during a manufacturing process of the analyte sensor.

In Example 42, the subject matter of any one or more of Examples 40-41 optionally includes the first sensor sensitivity being based on an additive combination of the first term and the second term.

In Example 43, the subject matter of any one or more of Examples 40-42 optionally includes the second term also being based on the calibration of the analyte sensor.

In Example 44, the subject matter of any one or more of Examples 31-43 optionally includes the first sensor sensitivity being based at least in part on a first term based on a calibration of the analyte sensor, a second term based on the property of the sensor signal, and a third term based on the calibration of the analyte sensor and the property of the sensor signal.

In Example 45, the subject matter of Example 44 optionally includes the first sensor sensitivity being based on an additive combination of the first term, the second term, and the third term.

Example 46 is an analyte sensor system for in vivo use, the analyte sensor system comprising: an analyte sensor; and sensor electronics in electrical communication with the analyte sensor, the sensor electronics being configured to perform operations comprising: determining that the analyte sensor is in a break-in period; and determining a first analyte concentration using a sensor signal generated by the analyte sensor after being inserted into a host and a break-in signal portion model.

In Example 47, the subject matter of Example 46 optionally includes the break-in signal portion model comprising a first portion describing a break-in sensor signal component and a second portion describing a hydration sensor signal component.

In Example 48, the subject matter of Example 47 optionally includes the first portion being based at least in part on a first distribution and the second portion being based on a second distribution different than the first distribution.

In Example 49, the subject matter of any one or more of Examples 46-48 optionally includes the determining that the analyte sensor is in the break-in comprising: accessing an indication that the analyte sensor has been inserted into a host; and determining that less than a threshold time has elapsed since the sensor has been inserted into the host.

Example 50 is a method of using an in vivo analyte sensor system, comprising: accessing non-enzyme sensor data generated by at least one non-enzyme sensor during a break-in period of the at least one non-enzyme sensor; using the non-enzyme sensor data to generate a break-in signal portion model; and using the break-in signal portion model and a sensor signal generated by an analyte sensor of the analyte sensor system to determine an estimated analyte concentration at a host.

In Example 51, the subject matter of Example 50 optionally includes before using the break-in signal portion model and a sensor signal generated by an analyte sensor of the analyte sensor system to determine an estimated analyte concentration at a host, determining that the analyte sensor is in a break-in period.

In Example 52, the subject matter of Example 51 optionally includes the determining that the analyte sensor is in break-in comprising: accessing an indication that the analyte sensor has been inserted into a host; and determining that less than a threshold time has elapsed since the sensor has been inserted into the host.

In Example 53, the subject matter of any one or more of Examples 50-52 optionally includes the break-in signal portion model comprising a first portion describing a break-in sensor signal component and a second portion describing a hydration sensor signal component.

In Example 54, the subject matter of Example 53 optionally includes the first portion being based at least in part on a first distribution and the second portion being based on a second distribution different than the first distribution.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments described in the present document.

FIG. 1 is a diagram showing one example of an environment including an analyte sensor system.

FIG. 2 is a diagram showing one example of a medical device system including the analyte sensor system of FIG. 1.

FIG. 3 is an illustration of an example analyte sensor.

FIG. 4 is an illustration of another example analyte sensor.

FIG. 5 is an enlarged view of an example analyte sensor portion.

FIG. 6 is a cross-sectional view of the analyte sensor of FIGS. 3 and 4.

FIG. 7 is a schematic illustration of a circuit that represents the behavior of an example analyte sensor.

FIG. 8 is an exploded side view showing one example of a sensor applicator.

FIG. 9A shows one example of the needle and sensor of FIG. 8 loaded prior to sensor insertion.

FIG. 9B shows one example of the needle and sensor after sensor insertion.

FIG. 9C shows one example of the sensor and needle during needle retraction.

FIG. 9D shows one example of the sensor remaining within the contact subassembly after needle retraction.

FIG. 10 is a perspective view of the sensor applicator of FIGS. 9A-D and a mounting unit according to one example including a safety latch mechanism.

FIG. 11 is a diagram showing another example of an analyte sensor applicator.

FIG. 12 is a diagram showing one example of an analyte sensor system including an analyte sensor and sensor electronics.

FIG. 13 is a diagram showing an example plot of sensor sensitivities of a group of continuous glucose sensors as a function of time during a sensor session.

FIG. 14 is a diagram showing three example plots of conversion functions at three different time periods of a sensor session.

FIG. 15 is a diagram showing one example of a plot of a sensor signal generated by an example analyte sensor after insertion into a host.

FIG. 16 is a flowchart showing one example of a process flow that may be executed in an analyte sensor system to determine a sensitivity of the analyte sensor based on a post-insertion sensor signal.

FIG. 17 is a flowchart showing another example of a process flow that may be executed in an analyte sensor system to determine a sensitivity of the analyte sensor based on a post-insertion sensor signal.

FIG. 18 is a flowchart showing another example of a process flow that may be executed in an analyte sensor system to determine a sensitivity of the analyte sensor based on a post-insertion sensor signal.

FIG. 19 is a flowchart showing another example of a process flow that may be executed in an analyte sensor system to measure a sensor signal after insertion.

FIG. 20 is a flowchart showing one example of a process flow that may be executed by an analyte sensor (e.g., sensor electronics thereof) to operate the analyte sensor during break-in utilizing a break-in signal portion model.

FIG. 21 is a block diagram illustrating a computing device hardware architecture, within which a set or sequence of instructions can be executed to cause a machine to perform examples of any one of the methodologies discussed herein.

### DETAILED DESCRIPTION

Various examples described herein are directed to analyte sensor systems and methods for using analyte sensor systems. An analyte sensor system includes an analyte sensor that is placed in contact with a bodily fluid of a host to measure a concentration of an analyte, such as glucose, in the bodily fluid. In some examples, the analyte sensor is inserted into the host to contact the bodily fluid in vivo. In some examples, the analyte sensor is inserted subcutaneously to contact interstitial fluid below the host's skin.

When the analyte sensor is exposed to analyte in the host's bodily fluid, an electrochemical reaction between the analyte sensor and the analyte causes the analyte sensor to generate a raw sensor signal that is indicative of the analyte concentration in the bodily fluid. For example, the analyte sensor may include two or more electrodes. Sensor electronics of the analyte sensor system apply a bias condition to the electrodes. The bias condition may be, for example, a potential difference applied between a working electrode of the analyte sensor and a reference electrode of the analyte sensor. The bias condition promotes the electrochemical reaction between the analyte and the analyte sensor, resulting in a current between the working electrode and at least one other analyte sensor electrode. The raw sensor signal may be, and/or may be based on, the current.

The sensor electronics uses the raw sensor signal to determine an estimated analyte value. In some examples, the sensor electronics is also programmed to output result data, which may include the estimated analyte value or other data. In some examples, the sensor electronics communicates result data to one or more other external devices.

For much of the useful life of the analyte sensor, the relationship between the raw sensor signal and the analyte concentration at the analyte sensor is linear, or approximately linear. The sensor electronics applies a slope, referred to as a sensor sensitivity, and an offset to the raw sensor signal to generate a corresponding analyte concentration. When an analyte sensor session begins, however, the linear relationship between the raw sensor signal and the analyte concentration does not hold. During a break-in or sensor break-in, the response of the analyte sensor to the analyte is nonlinear. As used herein, the term “break-in” or “sensor break-in” refers without limitation to a time required for the analyte sensor's raw sensor signal to provide a substantially linear response to the analyte concentration (e.g., glucose level).

Break-in may be caused by one or more factors. For example, when a bias potential is first applied to the analyte sensor, it promotes an electrochemical reaction with the analyte but may also promote other non-analyte electrochemical reactions, which also generate current at the analyte sensor, such as oxidation reactions at the electrode surfaces. The effects of non-analyte electrochemical reactions may decay with time but can initially make a substantial contribution to the raw sensor signal. The non-analyte electrochemical reactions contribute to electrochemical break-in. The term “electrochemical break-in” as used herein refers without limitation to a time after analyte sensor insertion in vitro and/or in vivo, at which the raw sensor signal from the analyte sensor settles to a substantially linear response to the analyte concentration after the application of the bias potential to the analyte sensor.

An analyte sensor membrane can also contribute to break-in. The membrane is also sometimes referred to as a membrane system. The membrane or membrane system, described in more detail herein, can perform functions in the analyte sensor including, for example, regulating the amount of analyte that reacts with the analyte sensor, providing an enzyme that reacts with the analyte, etc. In some examples, the analyte sensor is dry immediately before insertion into a host. When inserted in vitro or in vivo, the membrane is exposed to fluid (e.g., interstitial fluid) and begins to hydrate. As the membrane hydrates, its effects on the electrochemical reactions at the analyte sensor change, causing the raw sensor signal to behave differently. In some examples, the effects of the membrane become substantially constant when the membrane is fully hydrated. The term “membrane break-in” as used herein refers without limitation to a time taken for the membrane to equilibrate to its environment (e.g., the physiological environment in vivo).

Because the response of the analyte sensor to analyte is non-linear during break-in, it can be difficult to obtain usable measurements of analyte concentration during this time. The various examples described herein are directed to apparatuses, systems, and/or methods for shortening break-in and/or compensating the raw sensor signal during break-in to generate analyte concentration values.

Differences in sensor behavior during break-in can also be the result of other factors, such as differences in sterilization or other pre-insertion processing. For example, the sterilization techniques used to sterilize an analyte sensor can affect the way that the sensor behaves. As a result, analyte sensors sterilized using different sterilization techniques processes may exhibit different behaviors, including during break-in. This makes it challenging to obtain accurate estimated analyte concentrations during the break-in period across different sensors sterilized under different conditions.

In some examples, these and other challenges can be addressed by operating an analyte sensor based on properties exhibited by the analyte sensor shortly after insertion. In some examples, the post-insertion properties of the analyte sensor can be used to predict the behavior of the sensor, for example, during the break-in. Accordingly, sensor electronics may measure one or more post-insertion properties of the analyte sensor and generate a corresponding sensor sensitivity. The sensor electronics may use the sensor sensitivity determined using the post-insertion properties to generate estimated analyte concentration values, for example, during the break-in.

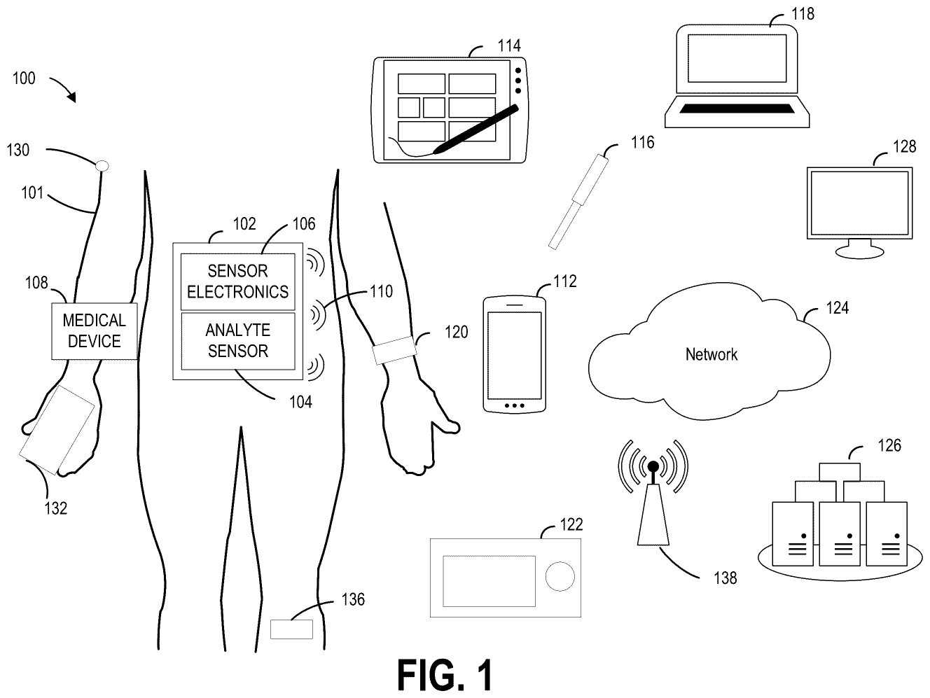

FIG. 1 is a diagram showing one example of an environment 100 including an analyte sensor system 102. The analyte sensor system 102 is coupled to a host 101, which may be a human patient. In some examples, the host is subject to a temporary or permanent diabetes condition or other health condition that makes analyte monitoring useful.

The analyte sensor system 102 includes an analyte sensor 104. In some examples, the analyte sensor 104 is or includes a glucose sensor configured to measure a glucose concentration in the host 101. The analyte sensor 104 can be exposed to analyte at the host 101 in any suitable way. In some examples, the analyte sensor 104 is fully implantable under the skin of the host 101. In other examples, the analyte sensor 104 is wearable on the body of the host 101 (e.g., on the body but not under the skin). Also, in some examples, the analyte sensor 104 is a transcutaneous device (e.g., with a sensor residing at least partially under or in the skin of a host). It should be understood that the devices and methods described herein can be applied to any device capable of detecting a concentration of an analyte, such as glucose, and providing an output signal that represents the concentration of the analyte.

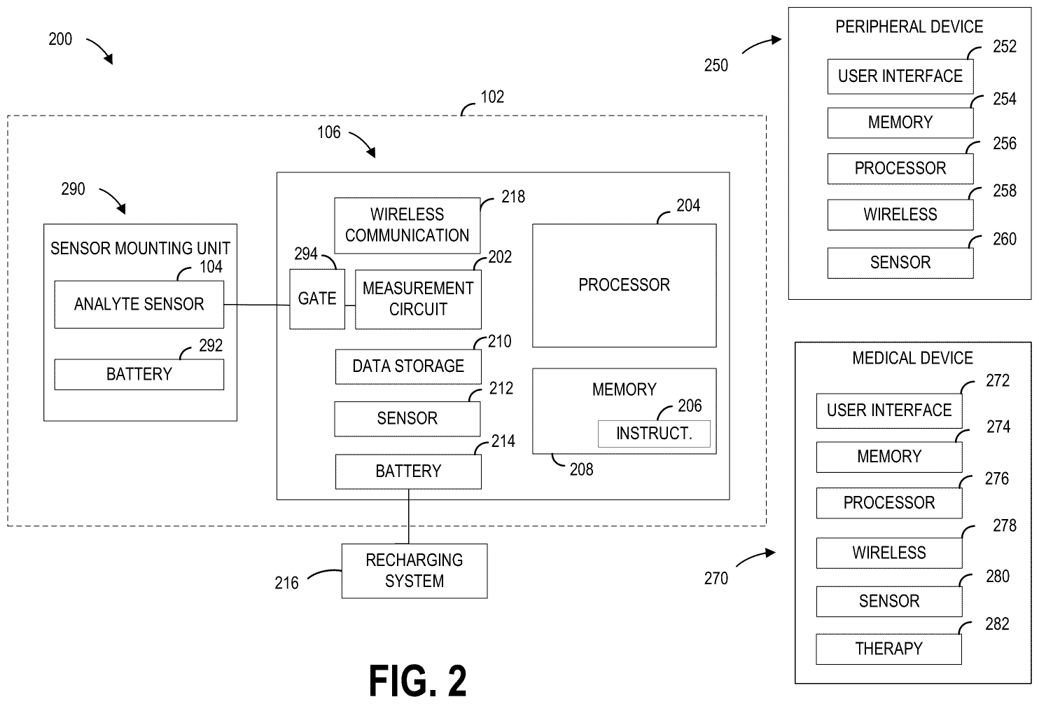

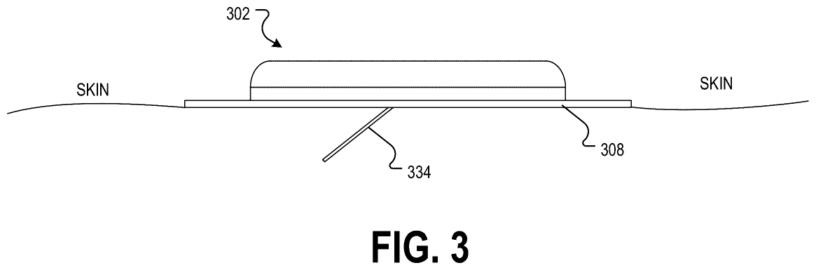

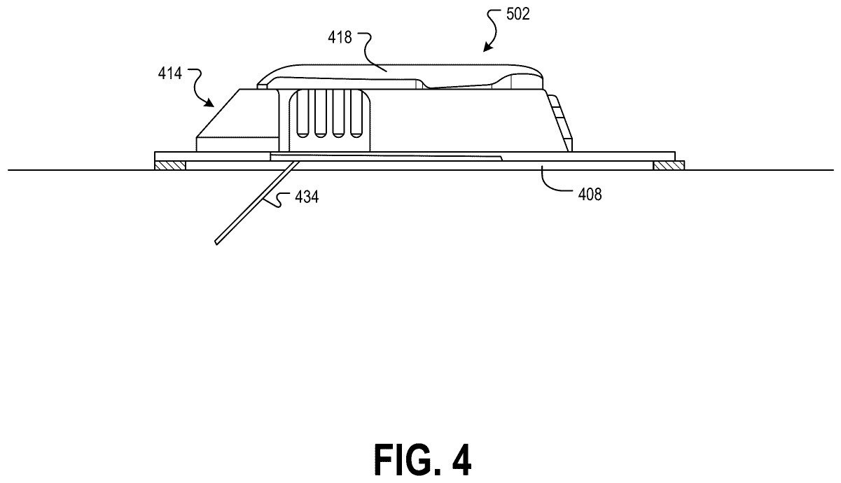

In the example of FIG. 1, the analyte sensor system 102 also includes sensor electronics 106. In some examples, the sensor electronics 106 and analyte sensor 104 are provided in a single integrated enclosure (See FIG. 3). In other examples, the analyte sensor 104 and sensor electronics 106 are provided as separate components or modules (See FIG. 4). For example, the analyte sensor system 102 may include a disposable (e.g., single-use) sensor mounting unit that may include the analyte sensor 104, a component for attaching the analyte sensor 104 to a host (e.g., an adhesive pad), and/or a mounting structure configured to receive a sensor electronics unit including some or all of the sensor electronics 106 shown in FIG. 2. The sensor electronics unit may be reusable.

The analyte sensor 104 may use any known method, including invasive, minimally-invasive, or non-invasive sensing techniques (e.g., optically excited fluorescence, microneedle, transdermal monitoring of glucose), to provide a raw sensor signal indicative of the concentration of the analyte in the host 101. The raw sensor signal may be converted into calibrated and/or filtered analyte concentration data used to provide a useful value of the analyte concentration (e.g., estimated blood glucose concentration level) to a user, such as the host 101 or a caretaker (e.g., a parent, a relative, a guardian, a teacher, a doctor, a nurse, or any other individual that has an interest in the wellbeing of the host 101).

In some examples, the analyte sensor 104 is or includes a continuous glucose sensor. A continuous glucose sensor can be or include a subcutaneous, transdermal (e.g., transcutaneous), and/or intravascular device. In some examples, such a sensor or device may recurrently (e.g., periodically, or intermittently) analyze sensor data. The glucose sensor may use any method of glucose measurement, including enzymatic, chemical, physical, electrochemical, spectrophotometric, polarimetric, calorimetric, iontophoretic, radiometric, immunochemical, and the like. In various examples, the analyte sensor system 102 may be or include a continuous glucose monitor sensor available from DexCom™, (e.g., the DexCom G5™ sensor, Dexcom G6™ sensor, the DexCom G7™ sensor, or any variation thereof), from Abbott™ (e.g., the Libre™ sensor), or from Medtronic™ (e.g., the Enlite™ sensor).

In some examples, analyte sensor 104 includes an implantable glucose sensor, such as described with reference to U.S. Pat. No. 6,001,067 and U.S. Patent Publication No. US-2005-0027463-A1, which are incorporated by reference. In some examples, analyte sensor 104 includes a transcutaneous glucose sensor, such as described with reference to U.S. Patent Publication No. US-2006-0020187-A1, which is incorporated by reference. In some examples, analyte sensor 104 may be configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007, all of which are incorporated by reference. In some examples, the continuous glucose sensor may include a transcutaneous sensor such as described in U.S. Pat. No. 6,565,509 to Say et al., which is incorporated by reference. In some examples, analyte sensor 104 may include a continuous glucose sensor that includes a subcutaneous sensor such as described with reference to U.S. Pat. No. 6,579,690 to Bonnecaze et al. or U.S. Pat. No. 6,484,046 to Say et al., which are incorporated by reference. In some examples, the continuous glucose sensor may include a refillable subcutaneous sensor such as described with reference to U.S. Pat. No. 6,512,939 to Colvin et al., which is incorporated by reference. The continuous glucose sensor may include an intravascular sensor such as described with reference to U.S. Pat. No. 6,477,395 to Schulman et al., which is incorporated by reference. The continuous glucose sensor may include an intravascular sensor such as described with reference to U.S. Pat. No. 6,424,847 to Mastrototaro et al., which is incorporated by reference.

The environment 100 may also include various other external devices including, for example, a medical device 108. The medical device 108 may be or include a drug delivery device such as an insulin pump or an insulin pen. In some examples, the medical device 108 includes one or more sensors, such as another analyte sensor, a heart rate sensor, a respiration sensor, a motion sensor (e.g., accelerometer), posture sensor (e.g., 3-axis accelerometer), acoustic sensor (e.g., to capture ambient sound or sounds inside the body). The medical device 108 may be wearable, e.g., on a watch, glasses, contact lens, patch, wristband, ankle band, or another wearable item, or may be incorporated into a handheld device (e.g., a smartphone). In some examples, the medical device 108 includes a multi-sensor patch that may, for example, detect one or more of an analyte levels (e.g., glucose, lactate, insulin, or other substance), heart rate, respiration (e.g., using impedance), activity (e.g., using an accelerometer), posture (e.g., using an accelerometer), galvanic skin response, tissue fluid levels (e.g., using impedance or pressure).

In some examples, the analyte sensor system 102 and the medical device 108 communicate with one another. Communication between the analyte sensor system 102 and medical device 108 may occur over any suitable wired connection and/or via a wireless communication signal 110. For example, the analyte sensor system 102 (e.g., the sensor electronics 106 thereof) may be configured to establish a communication connection with the medical device 108 using a suitable short-range communications medium such as, for example, a radio frequency medium (e.g., Bluetooth, Medical Implant Communication System (MICS), Wi-Fi, near field communication (NFC), radio frequency identification (RFID), Zigbee, Z-Wave or other communication protocols), an optical medium (e.g., infrared), a sonic medium (e.g., ultrasonic), a cellular protocol-based medium (e.g., Code Division Multiple Access (CDMA) or Global System for Mobiles (GSM)), and/or the like.

In some examples, the environment 100 also includes other external devices such as, for example, a wearable sensor 130. The wearable sensor 130 can include a sensor circuit (e.g., a sensor circuit configured to detect a glucose concentration or other analyte concentration) and a communication circuit, which may, for example, be an NFC circuit. In some examples, information from the wearable sensor 130 may be retrieved from the wearable sensor 130 using a user computing device 132, such as a smart phone, that is configured to communicate with the wearable sensor 130 via the wearable sensor's communication circuit, for example, when the user device 132 is placed near the wearable sensor 130. For example, swiping the user device 132 over the sensor 130 may retrieve sensor data from the wearable sensor 130 using NFC or other suitable wireless communication.

The use of NFC communication may reduce power consumption by the wearable sensor 130, which may reduce the size of a power source (e.g., battery or capacitor) in the wearable sensor 130 or extend the usable life of the power source. In some examples, the wearable sensor 130 may be wearable on an upper arm as shown. In some examples, a wearable sensor 130 may additionally or alternatively be on the upper torso of the patient (e.g., over the heart or over a lung), which may, for example, facilitate detecting heart rate, respiration, or posture. A wearable sensor 136 may also be on the lower body (e.g., on a leg) or other part of the body (e.g., on the abdomen).

In some examples, an array or network of sensors may be associated with the patient. For example, one or more of the analyte sensor system 102, and/or external devices, such as the medical device 108, wearable device 120 such as a watch, an additional wearable sensor 130 and/or the like, may communicate with one another via a short-range communication medium (e.g., Bluetooth, MICS, NFC, or any of the other options described above). The additional wearable sensor 130 may be any of the examples described above with respect to medical device 108. The analyte sensor system 102, medical device 108, and additional sensor 130 on the host 101 are provided for illustration and description and are not necessarily drawn to scale.

The environment 100 may also include one or more other external devices such as a hand-held smart device 112 (e.g., smart phone), tablet 114, smart pen 116 (e.g., insulin delivery pen with processing and communication capability), computer 118, a wearable device 120 such as a watch, or peripheral medical device 122 (which may be a proprietary device such as a proprietary user device available from DexCom™), any of which may communicate with the analyte sensor system 102 via a short-range communication medium, such as indicated by wireless communication signal 110, and may also communicate over a network 124 with a server system (e.g., remote data center) 126 or with a remote terminal 128 to facilitate communication with a remote user (not shown) such as a technical support staff member or a clinician.

The wearable device 120 may include an activity sensor, a heart rate monitor (e.g., light-based sensor or electrode-based sensor), a respiration sensor (e.g., acoustic- or electrode-based), a location sensor (e.g., GPS), or other sensors.

In some examples, the environment 100 includes a server system 126. The server system 126 can include one or more computing devices, such as one or more server computing devices. In some examples, the server system 126 is used to collect analyte data from the analyte sensor system 102 and/or analyte or other data from the plurality of other devices, and to perform analytics on collected data, generate, or apply universal or individualized models for glucose levels, and communicate such analytics, models, or information based thereon back to one or more of the devices in the environment 100. In some examples, the server system 126 gathers inter-host and/or intra-host break-in data to generate one or more break-in characteristics, as described herein.

The environment 100 may also include a wireless access point (WAP) 138 used to communicatively couple one or more of analyte sensor system 102, network 124, server system 126, medical device 108 or any of the peripheral devices described above. For example, WAP 138 may provide Wi-Fi and/or cellular connectivity within environment 100. Other communication protocols, such as NFC or Bluetooth, may also be used among devices of the environment 100.

FIG. 2 is a diagram showing one example of a medical device system 200 including the analyte sensor system 102 of FIG. 1. In the example of FIG. 2, the analyte sensor system 102 includes sensor electronics 106 and an example sensor mounting unit 290, although in some examples, it will be appreciated that the analyte sensor 104 and sensor electronics 106 may be included in a common enclosure. While a specific example of division of components between the sensor mounting unit 290 and sensor electronics 106 is shown, it is understood that some examples may include additional components in the sensor mounting unit 290 or in the sensor electronics 106, and that some of the components (e.g., a battery or supercapacitor) that are shown in the sensor electronics 106 may be alternatively or additionally (e.g., redundantly) provided in the sensor mounting unit 290.

In the example shown in FIG. 2, the sensor mounting unit 290 includes the analyte sensor 104 and a battery 292. In some examples, the sensor mounting unit 290 may be replaceable, and the sensor electronics 106 may include a debouncing circuit (e.g., gate with hysteresis or delay) to avoid, for example, recurrent execution of a power-up or power-down process when a battery is repeatedly connected and disconnected or avoid processing of a noise signal associated with removal or replacement of a battery.

The sensor electronics 106 may include electronics components that are configured to process sensor information, such as raw sensor signals, and generate corresponding analyte concentration values. The sensor electronics 106 may, for example, include electronic circuitry associated with measuring, processing, storing, or communicating continuous analyte sensor data, including prospective algorithms associated with processing and calibration of the raw sensor signal. The sensor electronics 106 may include hardware, firmware, and/or software that enables measurement of levels of the analyte via a glucose sensor. Electronic components may be affixed to a printed circuit board (PCB), or the like, and can take a variety of forms. For example, the electronic components may take the form of an integrated circuit (IC), such as an Application-Specific Integrated Circuit (ASIC), a microcontroller, and/or a processor.

In the example of FIG. 2, the sensor electronics 106 include a measurement circuit 202 (e.g., potentiostat) coupled to the analyte sensor 104 and configured to recurrently obtain analyte sensor readings using the analyte sensor 104. For example, the measurement circuit 202 may continuously or recurrently measure a raw sensor signal indicating a current flow at the analyte sensor 104 between a working electrode and a reference electrode. The sensor electronics 106 may include a gate circuit 294, which may be used to gate the connection between the measurement circuit 202 and the analyte sensor 104. For example, the analyte sensor 104 may accumulate charge over an accumulation period. After the accumulation period, the gate circuit 294 is opened so that the measurement circuit 202 can measure the accumulated charge. Gating the analyte sensor 104 may improve the performance of the sensor system 102 by creating a larger signal-to-noise or interference ratio (e.g., because charge accumulates from an analyte reaction, but sources of interference, such as the presence of acetaminophen near a glucose sensor, do not accumulate, or accumulate less than the charge from the analyte reaction).

The sensor electronics 106 may also include a processor 204. The processor 204 is configured to retrieve instructions 206 from memory 208 and execute the instructions 206 to control various operations in the analyte sensor system 102. For example, the processor 204 may be programmed to control application of bias potentials to the analyte sensor 104 via a potentiostat at the measurement circuit 202, interpret raw sensor signals from the analyte sensor 104, and/or compensate for environmental factors.

The processor 204 may also save information in data storage memory 210 or retrieve information from data storage memory 210. In various examples, data storage memory 210 may be integrated with memory 208, or may be a separate memory circuit, such as a non-volatile memory circuit (e.g., flash RAM). Examples of systems and methods for processing sensor analyte data are described in more detail herein and in U.S. Pat. Nos. 7,310,544 and 6,931,327.

The sensor electronics 106 may also include one or more sensors, such as the sensor 212, which may be coupled to the processor 204. The sensor 212 may be a temperature sensor, accelerometer, or another suitable sensor. The sensor electronics 106 may also include a power source such as a capacitor or battery 214, which may be integrated into the sensor electronics 106, or may be removable, or part of a separate electronics unit. The battery 214 (or other power storage component, e.g., capacitor) may optionally be rechargeable via a wired or wireless (e.g., inductive or ultrasound) recharging system 216. The recharging system 216 may harvest energy or may receive energy from an external source or on-board source. In various examples, the recharge circuit may include a triboelectric charging circuit, a piezoelectric charging circuit, an RF charging circuit, a light charging circuit, an ultrasonic charging circuit, a heat charging circuit, a heat harvesting circuit, or a circuit that harvests energy from the communication circuit. In some examples, the recharging circuit may recharge the rechargeable battery using power supplied from a replaceable battery (e.g., a battery supplied with a base component).

The sensor electronics 106 may also include one or more supercapacitors in the sensor electronics unit (as shown), or in the sensor mounting unit 290. For example, the supercapacitor may allow energy to be drawn from the battery 214 in a highly consistent manner to extend the life of the battery 214. The battery 214 may recharge the supercapacitor after the supercapacitor delivers energy to the communication circuit or to the processor 204, so that the supercapacitor is prepared for delivery of energy during a subsequent high-load period. In some examples, the supercapacitor may be configured in parallel with the battery 214. A device may be configured to preferentially draw energy from the supercapacitor, as opposed to the battery 214. In some examples, a supercapacitor may be configured to receive energy from a rechargeable battery for short-term storage and transfer energy to the rechargeable battery for long-term storage.

The supercapacitor may extend an operational life of the battery 214 by reducing the strain on the battery 214 during the high-load period. In some examples, a supercapacitor removes at least 10% of the strain off the battery during high-load events. In some examples, a supercapacitor removes at least 20% of the strain off the battery during high-load events. In some examples, a supercapacitor removes at least 30% of the strain off the battery during high-load events. In some examples, a supercapacitor removes at least 50% of the strain off the battery during high-load events.

The sensor electronics 106 may also include a wireless communication circuit 218, which may for example include a wireless transceiver operatively coupled to an antenna. The wireless communication circuit 218 may be operatively coupled to the processor 204 and may be configured to wirelessly communicate with one or more peripheral devices or other medical devices, such as an insulin pump or smart insulin pen.

In the example of FIG. 2, the medical device system 200 also includes optional external devices including, for example, a peripheral device 250. The peripheral device 250 may be any suitable user computing device such as, for example, a wearable device (e.g., activity monitor), such as a wearable device 120. In other examples, the peripheral device 250 may be a hand-held smart device (e.g., smartphone or other device such as a proprietary handheld device available from Dexcom), a tablet 114, a smart pen 116, or special-purpose computer 118 shown in FIG. 1.

The peripheral device 250 may include a user interface (UI) 252, a memory circuit 254, a processor 256, a wireless communication circuit 258, a sensor 260, or any combination thereof. The peripheral device 250 may not necessarily include all the components shown in FIG. 2. The peripheral device 250 may also include a power source, such as a battery.

The UI 252 may, for example, be provided using any suitable input/output device or devices of the peripheral device 250 such as, for example, a touch-screen interface, a microphone (e.g., to receive voice commands), or a speaker, a vibration circuit, or any combination thereof. The UI 252 may receive information from the host or another user (e.g., instructions, glucose values). The UI 252 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 256 may be configured to present information to a user, or receive input from a user, via the UI 252. The processor 256 may also be configured to store and retrieve information, such as communication information (e.g., pairing information or data center access information), user information, sensor data or trends, or other information in the memory circuit 254. The wireless communication circuit 258 may include a transceiver and antenna configured to communicate via a wireless protocol, such as any of the wireless protocols described herein. The sensor 260 may, for example, include an accelerometer, a temperature sensor, a location sensor, biometric sensor, or blood glucose sensor, blood pressure sensor, heart rate sensor, respiration sensor, or another physiologic sensor.

The peripheral device 250 may be configured to receive and display sensor information that may be transmitted by sensor electronics 106 (e.g., in a customized data package that is transmitted to the display devices based on their respective preferences). Sensor information (e.g., blood glucose concentration level) or an alert or notification (e.g., “high glucose level”, “low glucose level” or “fall rate alert” may be communicated via the UI 252 (e.g., via visual display, sound, or vibration). In some examples, the peripheral device 250 may be configured to display or otherwise communicate the sensor information as it is communicated from the sensor electronics 106 (e.g., in a data package that is transmitted to respective display devices). For example, the peripheral device 250 may transmit data that has been processed (e.g., an estimated analyte value level that may be determined by processing raw sensor data), so that a device that receives the data may not be required to further process the data to determine usable information (such as the estimated analyte value level). In other examples, the peripheral device 250 may process or interpret the received information (e.g., to declare an alert based on glucose values or a glucose trend). In various examples, the peripheral device 250 may receive information directly from sensor electronics 106, or over a network (e.g., via a cellular or Wi-Fi network that receives information from the sensor electronics 106 or from a device that is communicatively coupled to the sensor electronics 106).

In the example of FIG. 2, the medical device system 200 includes an optional medical device 270. For example, the medical device 270 may be an external device used in addition to or instead of the peripheral device 250. The medical device 270 may be or include any suitable type of medical or other computing device including, for example, the medical device 108, peripheral medical device 122, wearable device 120, wearable sensor 130, or wearable sensor 136 shown in FIG. 1. The medical device 270 may include a UI 272, a memory circuit 274, a processor 276, a wireless communication circuit 278, a sensor 280, a therapy circuit 282, or any combination thereof.

Similar to the UI 252, the UI 272 may be provided using any suitable input/output device or devices of the medical device 270 such as, for example, a touch-screen interface, a microphone, or a speaker, a vibration circuit, or any combination thereof. The UI 272 may receive information from the host or another user (e.g., glucose values, alert preferences, calibration coding). The UI 272 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 276 may be configured to present information to a user, or receive input from a user, via the UI 272. The processor 276 may also be configured to store and retrieve information, such as communication information (e.g., pairing information or data center access information), user information, sensor data or trends, or other information in the memory circuit 274. The wireless communication circuit 278 may include a transceiver and antenna configured communicate via a wireless protocol, such as any of the wireless protocols described herein.

The sensor 280 may, for example, include an accelerometer, a temperature sensor, a location sensor, biometric sensor, or blood glucose sensor, blood pressure sensor, heart rate sensor, respiration sensor, or another physiologic sensor. The medical device 270 may include two or more sensors (or memories or other components), even though only one sensor 280 is shown in the example in FIG. 2. In various examples, the medical device 270 may be a smart handheld glucose sensor (e.g., blood glucose meter), drug pump (e.g., insulin pump), or other physiologic sensor device, therapy device, or combination thereof.

In examples where medical device 270 is or includes an insulin pump, the pump and the analyte sensor system 102 may be in two-way communication (e.g., so the pump can request a change to an analyte transmission protocol, e.g., request a data point or request data on a more frequent schedule), or the pump and analyte sensor system 102 may communicate using one-way communication (e.g., the pump may receive analyte concentration level information from the analyte sensor system). In one-way communication, a glucose value may be incorporated in an advertisement message, which may be encrypted with a previously shared key. In a two-way communication, a pump may request a value, which the analyte sensor system 102 may share, or obtain and share, in response to the request from the pump, and any or all of these communications may be encrypted using one or more previously shared keys. An insulin pump may receive and track analyte (e.g., glucose) values transmitted from analyte sensor system 102 using one-way communication to the pump for one or more of a variety of reasons. For example, an insulin pump may suspend or activate insulin administration based on a glucose value being below or above a threshold value.

In some examples, the medical device system 200 includes two or more peripheral devices and/or medical devices that each receive information directly or indirectly from the analyte sensor system 102. Because different display devices provide many different user interfaces, the content of the data packages (e.g., amount, format, and/or type of data to be displayed, alarms, and the like) may be customized (e.g., programmed differently by the manufacturer and/or by an end user) for each device.

For example, referring now to the example of FIG. 1, a plurality of different peripheral devices may be in direct wireless communication with sensor electronics 106 (e.g., such as an on-skin sensor electronics 106 that are physically connected to the continuous analyte sensor 104) during a sensor session to enable a plurality of different types and/or levels of display and/or functionality associated with the displayable sensor information or to save battery power in the analyte sensor system 102, one or more specified devices may communicate with the analyte sensor system 102 and relay (i.e., share) information to other devices directly or through a server system (e.g., a network-connected data center) 126.

FIG. 3 is a side view of an example analyte sensor 334 that may be implanted into a host. An enclosure 302 may be adhered to the host's skin using an adhesive pad 308. The adhesive pad 308 may be formed from an extensible material, which may be removably attached to the skin using an adhesive. Sensor electronics may be positioned within the enclosure 302. The sensor 334 may extend from the enclosure 302 and under the skin of a host, as shown.

FIG. 4 is a side view of another example analyte sensor 434 in an arrangement including a mounting unit 414 and an electronics unit 418. The mounting unit 414 may be adhered to the host's skin using an adhesive pad 408, which may be like the adhesive pad 308 described herein. The electronics unit 418 comprises an enclosure 402 that may have sensor electronics positioned thereon. In some examples, the electronics unit 418 and mounting unit 414 are arranged in a manner like the sensor electronics 106 and sensor mounting unit 290 shown in FIGS. 1 and 2. For example, the sensor 434 may extend from the enclosure 402 via the mounting unit 414.

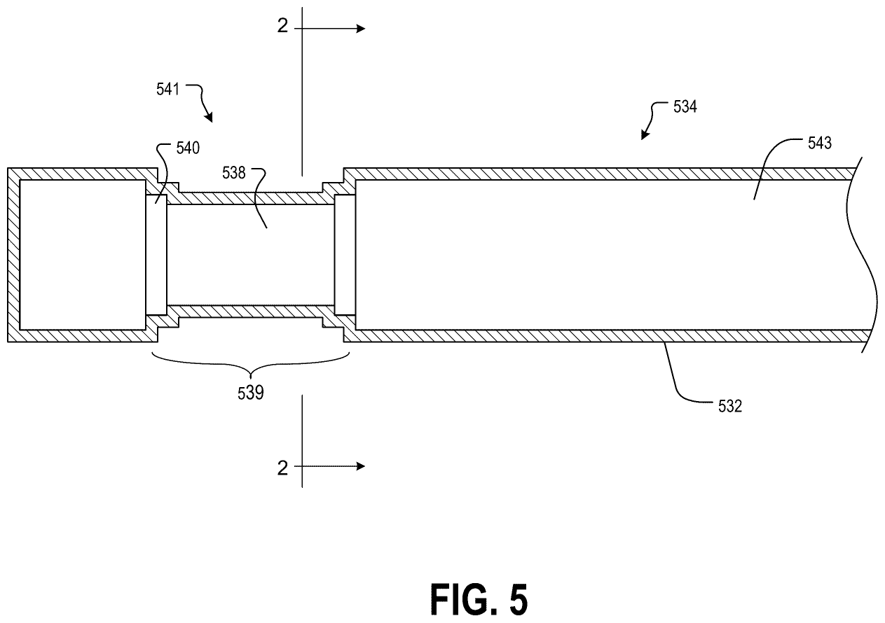

FIG. 5 is an enlarged view of a distal portion of an analyte sensor 534. The analyte sensor 534 illustrates one example arrangement that may be used to implement the analyte sensors described herein, such as, for example, the analyte sensors 104, 334, 434. The analyte sensor 534 may be adapted for insertion under the host's skin and may be mechanically coupled to an enclosure, such as the enclosures 402, and/or to a mounting unit, such as the mounting unit 414. The analyte sensor 534 may be electrically coupled to sensor electronics, which may be positioned within the enclosure 302, 402.

The example analyte sensor 534 shown in FIG. 5 includes an elongated conductive body 541. The elongated conductive body 541 can include a core with various layers positioned thereon. A first layer 538 at least partially surrounds the core and includes a working electrode, for example located in window 539. In some examples, the core and the first layer 538 are made of a single material (such as, for example, platinum). In some examples, the elongated conductive body 541 is a composite of two conductive materials, or a composite of at least one conductive material and at least one non-conductive material. A membrane system 532 is located over the working electrode and may cover other layers and/or electrodes of the sensor 534, as described herein.

The first layer 538 may be formed of a conductive material. The working electrode (at window 539) is an exposed portion of the surface of the first layer 538. Accordingly, the first layer 538 is formed of a material configured to provide a suitable electroactive surface for the working electrode. Examples of suitable materials include, but are not limited to, platinum, platinum-iridium, gold, palladium, iridium, graphite, carbon, a conductive polymer, an alloy, and/or the like.

A second layer 540 surrounds at least a portion of the first layer 538, thereby defining boundaries of the working electrode. In some examples, the second layer 540 serves as an insulator and is formed of an insulating material, such as polyimide, polyurethane, parylene, or any other suitable insulating materials or materials. In some examples, the second layer 540 is configured such that the working electrode (of the layer 538) is exposed via the window 539.

In some examples, the sensor 534 further includes a third layer 543 comprising a conductive material. The third layer 543 may comprise a reference electrode. In some examples, the third layer 543, including the reference electrode, is formed of a silver-containing material that is applied onto the second layer 540 (e.g., an insulator). The silver-containing material may include various materials and be in various forms such as, for example, Ag/AgCl-polymer pasts, paints, polymer-based conducting mixtures, inks, etc.

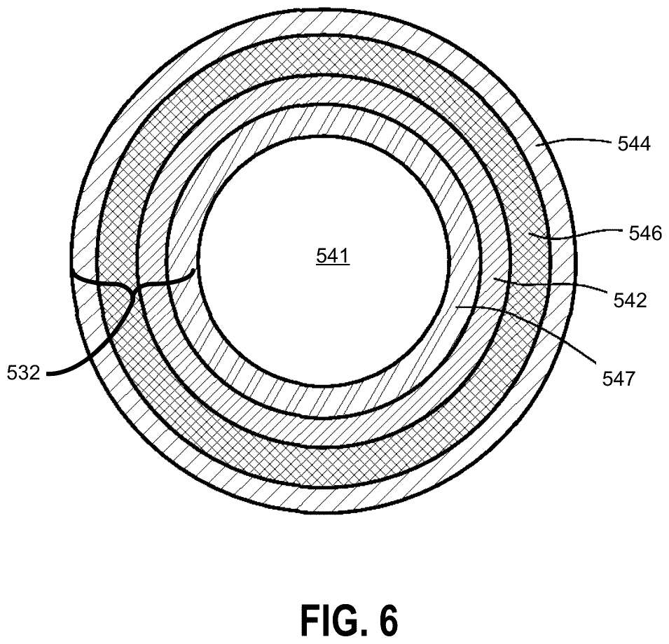

The analyte sensor 534 may include two (or more) electrodes, e.g., a working electrode at the layer 538 and exposed at window 539 and at least one additional electrode, such as a reference electrode of the layer 543. In the example arrangement of FIGS. 5-6, the reference electrode also functions as a counter electrode, although other arrangements can include a separate counter electrode. While the analyte sensor 534 may be used with a mounting unit in some examples, in other examples, the analyte sensor 534 may be used with other types of sensor systems. For example, the analyte sensor 534 may be part of a system that includes a battery and sensor in a single package, and may optionally include, for example, a near-field communication (NFC) circuit.

FIG. 6 is a cross-sectional view through the sensor 534 of FIG. 5 on plane 2-2 illustrating a membrane system 532. The membrane system 532 may include a number of domains (e.g., layers). In an example, the membrane system 532 may include an enzyme domain 542, a diffusion resistance domain 544, and a bioprotective domain 546 located around the working electrode. In some examples, a unitary diffusion resistance domain and bioprotective domain may be included in the membrane system 532 (e.g., wherein the functionality of both the diffusion resistance domain and bioprotective domain are incorporated into one domain).

The membrane system 532, in some examples, also includes an electrode layer 547. The electrode layer 547 may be arranged to provide an environment between the surfaces of the working electrode and the reference electrode that facilitates the electrochemical reaction between the electrodes. For example, the electrode layer 547 may include a coating that maintains a layer of water at the electrochemically reactive surfaces of the sensor 534.

In some examples, the sensor 534 may be configured for short-term implantation (e.g., from about 1 to 30 days). However, it is understood that the membrane system 532 can be modified for use in other devices, for example, by including only one or more of the domains, or additional domains. For example, a membrane system 532 may include a plurality of resistance layers, or a plurality of enzyme layers. In some examples, the resistance domain 544 may include a plurality of resistance layers, or the enzyme domain 542 may include a plurality of enzyme layers.

The diffusion resistance domain 544 may include a semipermeable membrane that controls the flux of oxygen and glucose to the underlying enzyme domain 542. As a result, the upper limit of linearity of glucose measurement is extended to a much higher value than that which is achieved without the diffusion resistance domain 544.

In some examples, the membrane system 532 may include a bioprotective domain 546, also referred to as a domain or biointerface domain, comprising a base polymer. However, the membrane system 532 of some examples can also include a plurality of domains or layers including, for example, an electrode domain, an interference domain, or a cell disruptive domain, such as described in more detail elsewhere herein and in U.S. Pat. Nos. 7,494,465, 8,682,608, and 9,044,199, which are incorporated herein by reference in their entirety.

It is to be understood that sensing membranes modified for other sensors, for example, may include fewer or additional layers. For example, in some examples, the membrane system 532 may comprise one electrode layer, one enzyme layer, and two bioprotective layers, but in other examples, the membrane system 532 may comprise one electrode layer, two enzyme layers, and one bioprotective layer. In some examples, the bioprotective layer may be configured to function as the diffusion resistance domain 544 and control the flux of the analyte (e.g., glucose) to the underlying membrane layers.

In some examples, one or more domains of the sensing membranes may be formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers.

In some examples, the sensing membrane can be deposited on the electroactive surfaces of the electrode material using known thin or thick film techniques (for example, spraying, electro-depositing, dipping, or the like). The sensing membrane located over the working electrode does not have to have the same structure as the sensing membrane located over the reference electrode; for example, the enzyme domain 542 deposited over the working electrode does not necessarily need to be deposited over the reference or counter electrodes.

Although the examples illustrated in FIGS. 5-6 involve circumferentially extending membrane systems, the membranes described herein may be applied to any planar or non-planar surface, for example, the substrate-based sensor structure of U.S. Pat. No. 6,565,509 to Say et al., which is incorporated by reference.

In an example in which the analyte sensor 534 is a glucose sensor, glucose analyte can be detected utilizing glucose oxidase. Glucose oxidase reacts with glucose to produce hydrogen peroxide (H₂O₂). The hydrogen peroxide reacts with the surface of the working electrode, producing two protons (2H⁺), two electrons (2e⁻) and one molecule of oxygen (O₂). This produces an electronic current that may be detected by the sensor electronics 106. The amount of current is a function of the glucose concentration level. A calibration curve may be used to provide an estimated glucose concentration level based on a measured current. The amount of current may also be a function of the diffusivity of glucose through the sensor membrane. The glucose diffusivity may change over time, which may cause the sensor glucose sensitivity to change over time, or “drift.”

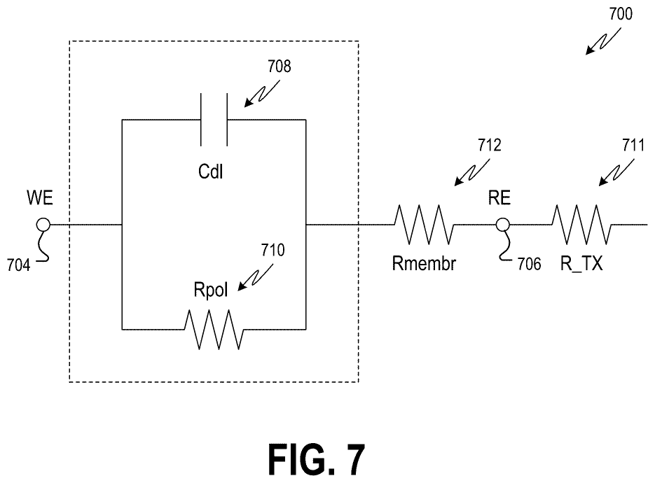

FIG. 7 is a schematic illustration of a circuit 700 that represents the behavior of an example analyte sensor, such as the analyte sensor 534 shown in FIGS. 5-6. As described above, the interaction of hydrogen peroxide (generated from the interaction between glucose analyte and glucose oxidase) and working electrode (WE) 704 produces a voltage differential between the working electrode (WE) 704 and reference electrode (RE) 706 which drives a current. The current may make up all or part of a raw sensor signal that is measured by the sensor electronics, such as the sensor electronics 106 of FIGS. 1-2, and used to estimate an analyte concentration (e.g., glucose concentration).

The circuit 700 also includes a double-layer capacitance (Cdl) 708, which occurs at an interface between the working electrode (WE) 704 and the adjacent membrane (not shown in FIG. 7, see, e.g., FIGS. 5-6 above). The double-layer capacitance (Cdl) may occur at an interface between the working electrode 704 and the adjacent membrane due to the presence of two layers of ions with opposing polarity, as may occur during application of an applied voltage between the working electrode 704 and reference electrode. The equivalent circuit 700 may also include a polarization resistance (Rpol) 710, which may be relatively large, and may be modeled, for example, as a static value (e.g., 100 mega-Ohms), or as a variable quantity that varies as a function of glucose concentration level.

An estimated analyte value may be determined from a raw sensor signal based upon a measured current (or charge flow) through the analyte sensor membrane 712 when a bias potential is applied to the sensor circuit 700. For example, sensor electronics or another suitable computing device can use the raw sensor signal and a sensitivity of the sensor, which correlates a detected current flow to a glucose concentration level, to generate the estimated analyte value. In some examples, the device also uses a break-in characteristic, as described herein.

With reference to the equivalent circuit 700, when a voltage is applied across the working and reference electrodes 704 and 706, a current may be considered to flow (forward or backward depending on polarity) through the internal electronics of transmitter (represented by R_Tx_internal) 711; through the reference electrode (RE) 706 and working electrode (WE) 704, which may be designed to have a relatively low resistance; and through the sensor membrane 712 (Rmembr, which is relatively small). Depending on the state of the circuit, current may also flow through, or into, the relatively large polarization resistance 710 (which is indicated as a fixed resistance but may also be a variable resistance that varies with the body's glucose level, where a higher glucose level provides a smaller polarization resistance), or into the double-layer capacitance 708 (i.e., to charge the double-layer membrane capacitor formed at the working electrode 704), or both.

The impedance (or conductance) of the membrane (Rmembr) 712 is related to electrolyte mobility in the membrane, which is in turn related to glucose diffusivity in the membrane. As the impedance goes down (i.e., conductance goes up, as electrolyte mobility in the membrane 712 goes up), the glucose sensitivity goes up (i.e., a higher glucose sensitivity means that a particular glucose concentration will produce a larger signal in the form of more current or charge flow). Impedance, glucose diffusivity, and glucose sensitivity are further described in U.S. Patent Publication No. US2012/0262298, which is incorporated by reference in its entirety.

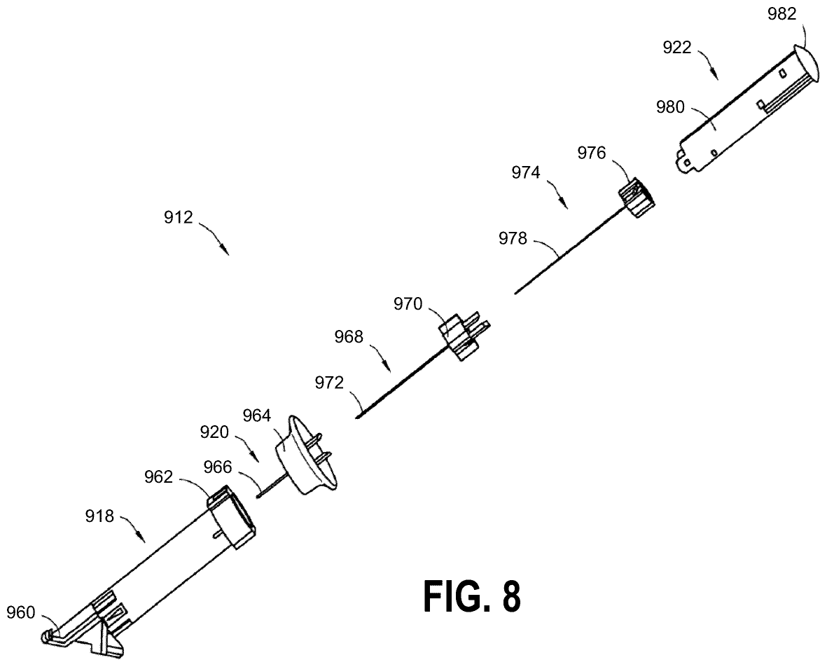

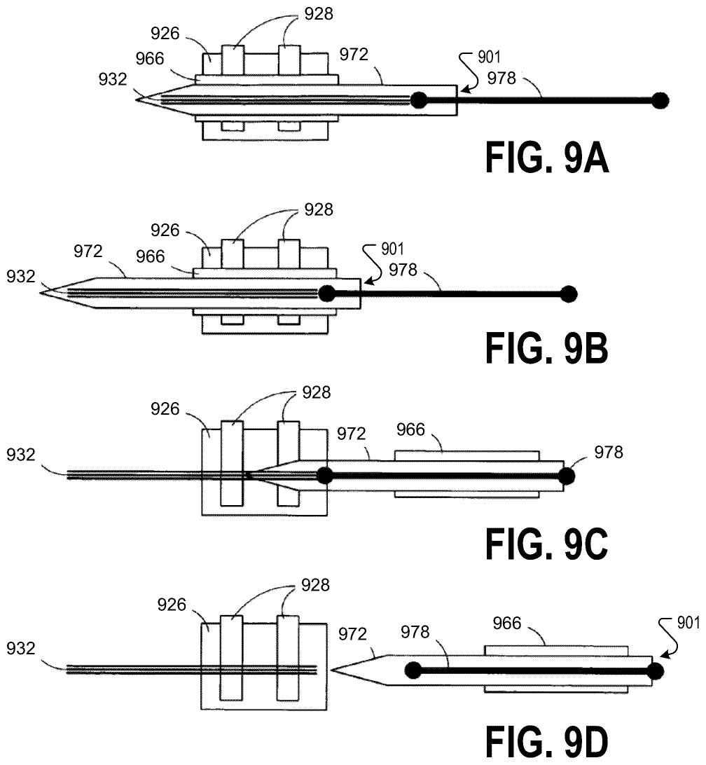

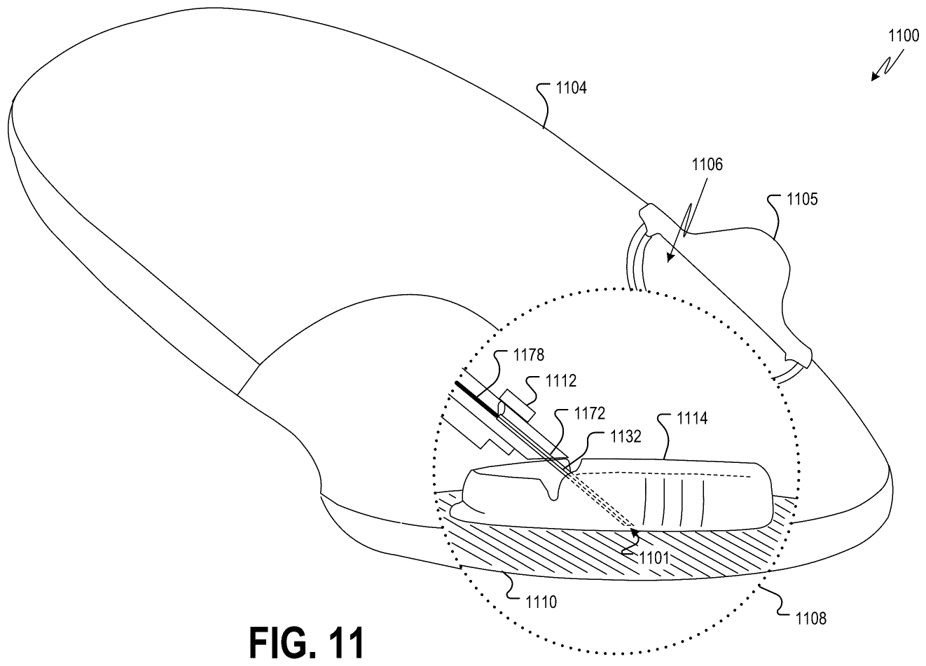

In various examples, an analyte sensor is packaged with a sensor applicator. FIGS. 8, 9A-D, 10 and 11 show example implementations of a sensor applicator. FIG. 8 is an exploded side view showing one example of a sensor applicator 912. In this example, the sensor applicator 912 includes an applicator body 918 that aides in aligning and guiding the sensor applicator components. The applicator body 918 includes an applicator body base 960 that matingly engages the mounting unit 914 (FIG. 11) and an applicator body cap 962 that enables appropriate relationships (for example, stops) between the sensor applicator components.

A guide tube subassembly 920 includes a guide tube carrier 964 and a guide tube 966. In some examples, the guide tube 966 is a cannula. The guide tube carrier 964 slides along the applicator body 918 and maintains the appropriate relative position of the guide tube 966 during insertion and subsequent retraction. For example, prior to and during insertion of the sensor, the guide tube 966 extends through the contact subassembly 926 to maintain an opening that enables easy insertion of the needle therethrough (see FIGS. 9A to 9D). During retraction of the sensor, the guide tube subassembly 920 is pulled back, engaging with, and causing the needle and associated moving components to retract back into, the sensor applicator 912 (See FIGS. 9C and 9D). In some examples, a lubricant (e.g., petroleum jelly) is placed within the contact subassembly 926 such that it surrounds the guide tube 966 (e.g., cannula), thereby allowing the guide tube 966 to easily retract back into the sensor applicator 912, for example, without causing compression or deformation in the contact subassembly 926.

A needle subassembly 968 is provided that includes a needle carrier 970 and needle 972. The needle carrier 970 cooperates with the other sensor applicator components and carries the needle 972 between its extended and retracted positions. The needle 972 can be of any appropriate size that can encompass an analyte sensor 932 (FIGS. 9A-9D) and aid in its insertion into the host. Example sizes include from about 32 gauge or less to about 18 gauge or more, more preferably from about 28 gauge to about 25 gauge, to provide a comfortable insertion for the host. Referring to the inner diameter of the needle, approximately 0.006 inches to approximately 0.023 inches may be used, and 0.013 inches may also be used. The needle carrier 970 is configured to engage with the guide tube carrier 964, while the needle 972 is configured to slidably nest within the guide tube 966, which allows for easy guided insertion (and retraction) of the needle through the contact subassembly 926.

A push rod subassembly 974 is provided that includes a push rod carrier 976 and a push rod 978. The push rod carrier 976 cooperates with other sensor applicator components to ensure that the analyte sensor 932 is properly inserted into the host's skin, namely the push rod carrier 976 carries the push rod 978 between its extended and retracted positions. In this example, the push rod 978 is configured to slidably nest within a cannula 901 of the needle 972, which allows for the analyte sensor 932 to be pushed (released) from the needle 972 upon retraction of the needle 972. This is described in more detail with reference to FIGS. 9A-9D. In some examples, a slight bend or serpentine shape is designed into or allowed in the sensor in order to maintain the sensor within the needle 972 by interference. While not wishing to be bound by theory, it is believed that a slight friction fit of the analyte sensor 932 within the needle 972 may minimize motion of the analyte sensor 932 during withdrawal of the needle 972 and maintain the analyte sensor 932 within the needle prior to withdrawal of the needle 972.

A plunger subassembly 922 is provided that includes a plunger 980 and plunger cap 982. The plunger subassembly 922 cooperates with other sensor applicator components to ensure proper insertion and subsequent retraction of the needle 972. In this example, the plunger 980 is configured to engage with the push rod 978 to ensure the analyte sensor 932 remains extended (namely, in the host) during retraction, such as is described in more detail with reference to FIG. 9C.

FIGS. 9A through 9D are schematic side cross-sectional views that illustrate the applicator components and their cooperating relationships at various stages of sensor insertion. FIG. 9A shows one example of the needle 972 and analyte sensor 932 loaded prior to sensor insertion. FIG. 9B shows one example of the needle 972 and analyte sensor 932 after sensor insertion. FIG. 9C shows one example of the analyte sensor 932 and needle 972 during needle retraction. FIG. 9D shows one example of the analyte sensor 932 remaining within the contact subassembly 926 after needle retraction. Although the examples of FIGS. 8, 9A-9D and 11 suggest manual insertion and/or retraction of the various components, automation of one or more of the stages can also be employed. For example, spring-loaded mechanisms can be triggered to automatically insert and/or retract the sensor, needle, or other cooperative applicator components can be implemented.

Referring to FIG. 9A, the analyte sensor 932 is shown disposed within the needle 972, which is disposed within the guide tube 966. In this example, the guide tube 966 is provided to maintain an opening within the contact subassembly 926 and/or contacts 928 to provide minimal friction between the needle 972 and the contact subassembly 926 and/or contacts 928 during insertion and retraction of the needle 972. However, the guide tube 966 is an optional component, which can be advantageous in some examples where the contact subassembly 926 and/or the contacts 928 are formed from an elastomer or other material with a relatively high-friction coefficient. The guide tube 966 can be omitted, for example, in other examples in which the contact subassembly 926 and/or the contacts 928 are formed from a material with a relatively low-friction coefficient (for example, hard plastic or metal). A guide tube 966, or the like, may be advantageous in examples in which the contact subassembly 926 and/or the contacts 928 are formed from a material designed to frictionally hold the analyte sensor 932 (see FIG. 9D), for example, by the relaxing characteristics of an elastomer, or the like. In these examples, the guide tube 966 may be provided to ease insertion of the needle 972 through the contacts 928, while allowing for a frictional hold of the contacts 928 on the analyte sensor 932 upon subsequent needle retraction. Stabilization of the analyte sensor 932 in or on the contacts 928 is described in more detail with reference to FIG. 9D. Although FIG. 9A illustrates the needle 972 and analyte sensor 932 inserted into the contacts subassembly 926 as the initial loaded configuration, alternative examples contemplate a step of loading the needle 972 through the guide tube 966 and/or contacts 928 prior to sensor insertion.

Referring to FIG. 9B, the analyte sensor 932 and needle 972 are shown in an extended position. In this stage, the push rod 978 has been forced to a forward position, for example by pushing on the plunger shown in FIG. 7, or the like. The plunger 980 (FIG. 8) is designed to cooperate with other of the sensor applicator components to ensure that analyte sensor 932 and the needle 972 extend together to a forward position (as shown). For example, the push rod 978 may be designed to cooperate with other of the sensor applicator components to ensure that the analyte sensor 932 maintains the forward position simultaneously within the needle 972.

Referring to FIG. 9C, the needle 972 is shown during the retraction process. In this stage, the push rod 978 is held in its extended (forward) position in order to maintain the analyte sensor 932 in its extended (forward) position until the needle 972 has substantially fully retracted from the contacts 928. Simultaneously, the cooperating sensor applicator components retract the needle 972 and guide tube 966 backward by a pulling motion (manual or automated) thereon. In some examples, the guide tube carrier 964 (FIG. 8) engages with cooperating applicator components such that a backward (retraction) motion applied to the guide tube carrier retracts the needle 972 and guide tube 966, without (initially) retracting the push rod 978. In an alternative example, the push rod 978 can be omitted and the analyte sensor 932 held in its forward position by a cam, elastomer, or the like, which is in contact with a portion of the sensor while the needle moves over another portion of the sensor. One or more slots can be cut in the needle to maintain contact with the sensor during needle retraction.

Referring to FIG. 9D, the needle 972, guide tube 966, and push rod 978 are all retracted from contact subassembly 926, leaving the analyte sensor 932 disposed therein. The cooperating sensor applicator components are designed such that when the needle 972 has substantially cleared from the contacts 928 and/or contact subassembly 926, the push rod 978 is retracted along with the needle 972 and guide tube 966. The sensor applicator 912 can then be released (manually or automatically) from the contacts 928.

In various examples, the contacts 928 are elastomeric contacts to ensure a retention force that retains the analyte sensor 932 within the mounting unit and to ensure stable electrical connection of the analyte sensor 932 and its associated contacts 928. Although the illustrated examples and associated text describe the analyte sensor 932 extending through the contacts 928 to form a friction fit therein, a variety of alternatives are contemplated. In some examples, the analyte sensor 932 is configured to be disposed adjacent to the contacts 928 (rather than between the contacts 928). The contacts 928 can be constructed in a variety of known configurations, for example, metallic contacts, cantilevered fingers, pogo pins, or the like, which are configured to press against the sensor after needle retraction.

The illustrated examples are designed with coaxial contacts 928; namely, the contacts 928 are configured to contact the working and reference electrodes of the analyte sensor 932 axially along a distal portion of the analyte sensor 932. For example, the working electrode of the analyte sensor 932 may extend farther than the reference electrode, which allows coaxial connection of the electrodes with the contacts 928 at locations spaced along the distal portion of the sensor.

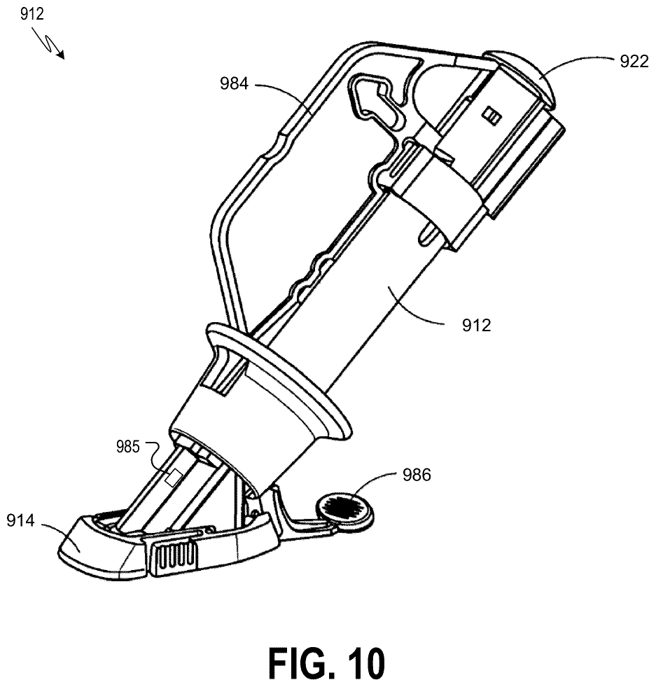

FIG. 10 is a perspective view of a sensor applicator 912 and mounting unit 914 according to one example including a safety latch mechanism 984. Although FIG. 10 shows the sensor applicator 912 engaged with a sensor mounting unit 914, it will be appreciated that the sensor applicator 912 may be configured to engage instead with an enclosure, such as the enclosure 302 described with respect to FIG. 3.

In the example of FIG. 10, the safety latch mechanism 984 is configured to lock the plunger subassembly 922 in a stationary position such that it cannot be accidentally pushed prior to release of the safety latch mechanism 984. In this example, the analyte sensor 932 is preferably packaged (e.g., shipped) in this locked configuration, where the safety latch mechanism 984 holds the plunger subassembly 922 in its extended position. This may prevent the analyte sensor 932 from being prematurely inserted (e.g., accidentally released). The safety latch mechanism 984 may be configured such that a pulling force shown in the direction of the arrow (see FIG. 10) releases the lock of the safety latch mechanism 984 on the plunger subassembly 922, thereby allowing sensor insertion. Although one safety latch mechanism 984 that locks the plunger subassembly 922 is illustrated and described herein, a variety of safety latch mechanism configurations that lock the sensor to prevent it from prematurely releasing (i.e., that lock the sensor prior to release of the safety latch mechanism 984) are contemplated, as can be appreciated by one skilled in the art.

FIG. 10 additionally illustrates a force-locking mechanism 986 included in certain examples of the sensor system, wherein the force-locking mechanism 986 is configured to ensure a proper mate between an electronics unit (e.g., electronics unit 418 of FIG. 4) and the mounting unit 914. In some circumstances, it can be advantageous to ensure the electronics unit has been properly mated (e.g., snap-fit or sealingly mated) to the mounting unit. Accordingly, upon release of the sensor applicator 912 from the mounting unit 914 after sensor insertion, the force-locking mechanism 986 allows the user to ensure a proper mate and/or seal therebetween.

In practice, a user pivots (e.g., lifts or twists) the force-locking mechanism such that it provides force on the electronics unit (such as electronics unit 418 of FIG. 4) by pulling up on the circular tab illustrated in FIG. 10. The force-locking mechanism is preferably released thereafter. Although one system and one method for providing a secure and/or sealing fit between the electronics unit and the mounting unit are illustrated, various other force-locking mechanisms can be employed that utilize a variety of systems and methods for providing a secure and/or sealing fit between the electronics unit and the mounting unit (housing).

In some examples, the sensor applicator 912 shown in FIGS. 8, 9A-9D and 10 can also comprise a transmitter 985. The transmitter 985 is configured to generate a wireless signal that may be detected by the sensor electronics 106, as described herein. The wireless signal generated by the transmitter 985 may be in a first state or undeployed state before the sensor applicator 912 inserts the sensor. After and/or as the sensor is inserted, the transmitter 985 may modify the wireless signal to a second state or deployed state. The sensor electronics may detect the change in state of the wireless signal to determine whether to transition from a sleep mode to an active mode, as described herein.

The transmitter 985 may be of any suitable design that generates a wireless signal having at least two states. In some examples, the transmitter 985 is an electromagnet or permanent magnet and the wireless signal is a magnetic field generated by the transmitter 985. One state of the wireless signal may be the presence of the magnetic field (e.g., the magnetic field having a field strength greater than a threshold). Another state of the wireless signal may be the absence of the magnetic field (e.g., the magnetic field having a field strength less than a threshold). The presence of the magnetic field may indicate the undeployed state and the absence of the magnetic field may indicate the deployed state, or vice versa.

In some examples in which the transmitter 985 includes an electromagnet and/or permanent magnet, the first state of the wireless signal may correspond to a first magnetic polarity of the magnetic field and the second state of the wireless signal may correspond to a second magnetic polarity of the magnetic field, which may be opposite the first polarity. One polarity of the magnetic field may correspond to the undeployed state and another polarity of the magnetic field may correspond to the deployed state. The transmitter 985 may transition the wireless signal from the first state to the second state, for example, by reversing the direction of current provided to an electromagnet and/or reversing the physical orientation of the magnet and/or the like.

Other suitable transmitter arrangements may also be used to modify the state of the wireless signal in other ways. In some examples, the transmitter 985 is or includes an optical transmitter configured to generate an optical signal. Also, in some examples, the transmitter 985 is or includes a sonic transmitter configured to generate a sonic signal. In some examples, the transmitter 985 is or includes a radio frequency transmitter, such as an RFID transmitter.

FIG. 11 is a diagram showing another example of an analyte sensor applicator 1100. In the example of FIG. 11, the sensor applicator 1100 includes an applicator enclosure 1104. The sensor applicator 1100 is automated and, for example, includes a spring-loaded mechanism for initiating sensor insertion (not shown in FIG. 11). For example, the spring-loaded mechanism may be actuated by depressing an insertion button 1106. A safety latch mechanism 1105 is positioned over the insertion button 1106 to prevent accidental insertion of the analyte sensor 1132. The safety latch mechanism 1105 can be removed, for example, by pulling it away from the applicator enclosure 1104.

FIG. 11 includes a window 1108 showing components that are inside of the applicator enclosure 1104 including, for example, a needle 1172 and a mounting unit 1114. The mounting unit 1114 is coupled to an example adhesive pad 1110 for adhering the mounting unit 1114 to the skin of a host. In examples that do not include a separate electronics unit, such as the example of FIG. 3, the enclosure 302 and associated adhesive pad 308 may be positioned in a manner similar to that of the mounting unit 1114 and adhesive pad 1110.

In the example of FIG. 11, a push rod 1178 may operate in a manner similar to that of the push rod 978 described above to push the analyte sensor 1132 into the host. A lumen 1101 of the needle 1172 can include a hydrating agent, for example, as described herein. The example of FIG. 11, the applicator 1100 also includes a transmitter 1112 which may be arranged, for example, in a manner similar to that of the transmitter 985 of FIG. 10.

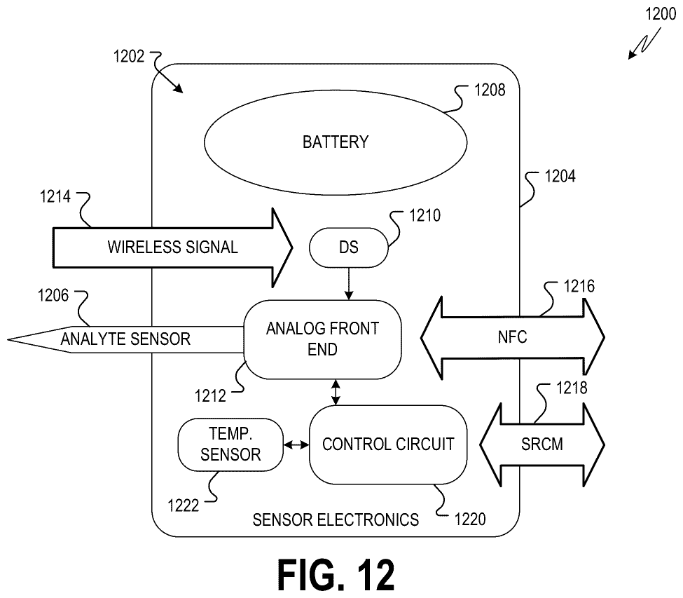

FIG. 12 is a diagram showing one example of an analyte sensor system 1200 including an analyte sensor 1206 and sensor electronics 1202. The analyte sensor 1206 may be arranged, for example, as described herein with respect to FIGS. 3-6 and/or according to any other suitable arrangement such as, for example a planar sensor arrangement.

The sensor electronics 1202 are positioned within an enclosure 1204. The analyte sensor 1206 extends from the enclosure 1204 as shown. In some examples, the analyte sensor 1206 is mechanically coupled to the enclosure 1204, for example, as illustrated in FIG. 3. In some examples, the analyte sensor 1206 is mechanically coupled to a sensor mounting unit, as described herein.

The analyte sensor 1206 may be in electrical communication with an analog front end 1212 of the sensor electronics 1202. In examples in which the analyte sensor 1206 is mechanically coupled to the enclosure 1204, the analyte sensor 1206 may extend through the enclosure 1204 in a sealed manner and be in direct electrical contact with the analog front end 1212. In examples in which the analyte sensor 1206 is mechanically coupled to a sensor mounting unit, an electrical connection between the analyte sensor 1206 and the analog front end 1212 may be via a connector between the sensor mounting unit and the enclosure 1204.

The analog front end 1212 may be configured to receive analog electrical signals from various components of the sensor electronics 1202 and provide corresponding digital electrical signals to a control circuit 1220. For example, as described, the analyte sensor 1206 may be in electrical communication with the analog front end 1212 to provide an analog raw sensor signal to the analog front end 1212. The analog front end 1212 may comprise various amplifiers, filters, conditioners, analog-to-digital converters, and/or the like to condition the raw sensor signal and covert it to a digital raw sensor signal, which may be provided to the control circuit 1220. The control circuit 1220 may be configured to convert the digital raw sensor signal to an estimated analyte value, which may be output as described herein.

The analog front end 1212 may also be in communication with a deployment sensor 1210. The deployment sensor 1210 receives a wireless signal 1214. The wireless signal 1214 may be generated by a transmitter at an applicator, such as, for example, the transmitter 1112 of the applicator 1100 and/or the transmitter 985 of the applicator 912. The deployment sensor 1210 may be configured to detect the wireless signal 1214 and generate an output indicative of the state of the wireless signal 1214. In examples where the wireless signal 1214 is a magnetic signal, the deployment sensor 1210 may be or comprise a magnetic sensor, such as a Tunnel Magento-Resistance effect (TMR) sensor. In examples where the wireless signal 1214 is an optical signal, the deployment sensor 1210 may be or comprise a photoresistor or another optical sensor. In examples where the wireless signal 1214 is a radio frequency (RF) or other similar signal, the deployment sensor 1210 may comprise an antenna and/or other RF receiver components. The analog front end 1212 may receive an output of the deployment sensor 1210 and provide the output to the control circuit 1220 for further processing. In some examples, as shown in FIG. 12, the analog front end 1212 and control circuit 1220 may be coupled to the analyte sensor 1206 prior to deployment. In this way, the analog front end 1212 and control circuit 1220 may detect deployment of the analyte sensor 1206 (e.g., via wireless signal 1214) close in time to the actual insertion of the analyte sensor 1206.

In the example of FIG. 12, the analog front end 1212 comprises circuitry for receiving and transmitting near field communication (NFC) signals 1216. NFC signals 1216 may be utilized to communicate with various external devices. In some examples, the wireless signal 1214 may be implemented as an NFC signal 1216.

The control circuit 1220 is configured to receive digital signals from the analog front end 1212 and perform various processing on the signals. For example, the control circuit 1220 may be and/or include a microcontroller or other processor. One or more processers of the control circuit 1220 may be programmed to execute software instructions for executing various operations, for example, as described herein. The software instructions may be stored at a data storage that may be part of the control circuit 1220 or may be implemented at a different location.

In some examples, the control circuit 1220 converts a raw sensor signal (e.g., a digital raw sensor signal) to a corresponding estimated analyte value. The control circuit 1220 may provide result data including the estimated analyte value as an output, for example, in the manner described herein. In the example of FIG. 12, the control circuit 1220 is in communication with a temperature sensor 1222, for example, as described herein. In this example, the temperature sensor 1222 is a digital sensor. In other examples, an analog temperature sensor (or other sensor, as described herein) may be in communication with the control circuit 1220 via the analog front end 1212. In some examples, the control circuit 1220, and/or other component of the sensor electronics 1202, is also configured to communicate with one or more external devices via a short-range wireless communication medium 1218 such as, for example, Bluetooth®, Bluetooth LE® and/or the like.

The control circuit 1220 may also be programmed to transition the analyte sensor system 1200 from a sleep mode to an active mode. For example, various components of the sensor electronics 1202 are powered by a battery 1208. In some examples, the battery 1208 is installed at the time that the analyte sensor system 1200 is manufactured. In some examples, the analyte sensor system 1200 is configured to operate for its full lifecycle on a single charge of the battery 1208. Accordingly, the analyte sensor system 1200 may be configured in a sleep mode after manufacturing. In the sleep mode, the sensor electronics 1202 maintains the analyte sensor system 1200 in an arrangement for low power consumption. For example, the non-essential components of the analyte sensor system 1200 may be switched off. Also, for example, operations executed by the sensor electronics 1202 may be minimized to save power. The control circuit 1220 may be programmed to maintain the sleep mode until the analyte sensor 1206 is inserted in vivo into a host and a sensor session is to begin.

When the control circuit 1220, or other component of the sensor electronics 1202, determines that a sensor session is to begin, the control circuit 1220, or other component of the sensor electronics 1202, transitions the analyte sensor system 1200 into the active mode. This may include, for example, applying a bias condition to the analyte sensor, using the raw sensor signal (e.g., a digital raw sensor signal) to determine and output an estimated analyte concentration, and/or the like. The sensor electronics 1202 may determine to transition the analyte sensor system 1200 to the active mode, for example, using the wireless signal 1214 and/or the raw sensor signal provided by the analyte sensor 1206. Although specific circuitry, components, and interconnections are illustrated in the example analyte sensor system 1200 shown in FIG. 12, alternative examples may incorporate additional, fewer, and/or alternative circuitry, components, and/or interconnections.

### Determination of Sensor Sensitivity

As described elsewhere herein, in certain examples, self-calibration of the analyte sensor system can be performed by determining sensor sensitivity based on a sensitivity profile (and a measured or estimated baseline), so that Equation [1] can be solved:

$$y = mx + b \qquad [ 1 ]$$

In Equation [1], y represents the sensor signal, x represents the estimated glucose concentration (mg/dL), m represents the sensor sensitivity to the analyte (counts/mg/dL), and b represents the baseline signal. The sensor signal y and baseline b are based on a current generated by the analyte sensor. For example, the sensor signal y and baseline b may be expressed in Amps (e.g., milliamps or microamps) or in counts generated by a current sensor of the sensor electronics. In some examples, Equation [1] can be used to form a conversion function for converting the sensor signal into an estimated glucose or other analyte concentration.

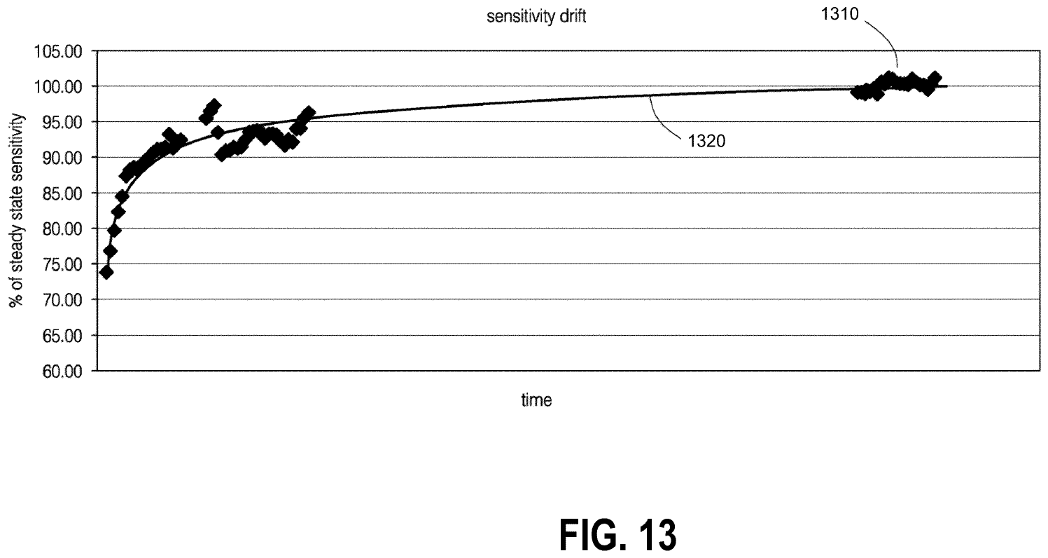

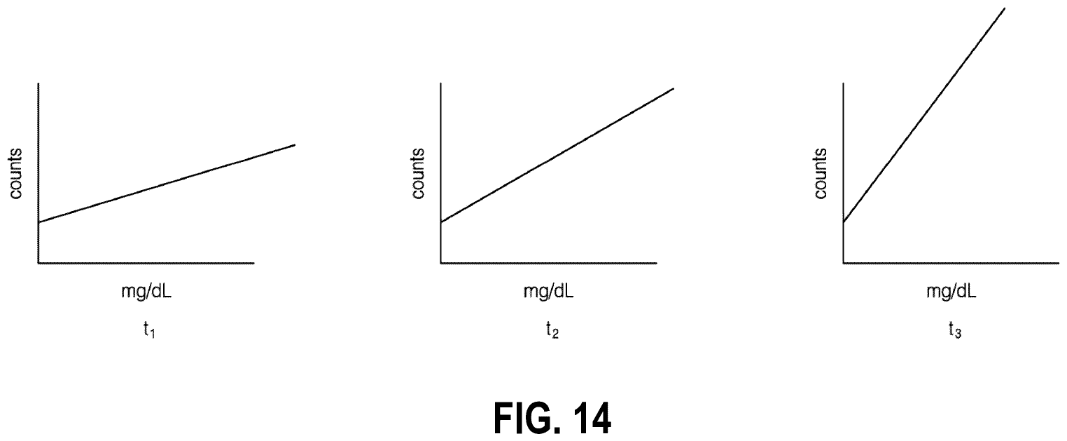

In some examples, it has been found that a sensor's sensitivity to analyte concentration during a sensor session will change or drift as a function of time. FIG. 13 illustrates this phenomenon and provides a plot of sensor sensitivities 1310 of a group of continuous glucose sensors as a function of time during a sensor session. FIG. 14 provides three plots of conversion functions at three different time periods of a sensor session. As shown in FIG. 14, the three conversion functions have different slopes, each of which correspond to a different sensor sensitivity. Accordingly, the differences in slopes over time illustrate that changes or drift in sensor sensitivity occur over a sensor session.

Referring back to the study associated with FIG. 13, the sensors were made in substantially the same way under substantially the same conditions. The sensor sensitivities associated with the y-axis of the plot are expressed as a percentage of a substantially steady state sensitivity that was reached about three days after start of the sensor session. In addition, these sensor sensitivities correspond to measurements obtained from analyte concentration tests performed using a glucometer or other suitable device. As shown in the plot, the sensitivities (expressed as a percentage of a steady state sensitivity) of each sensor, as measured, are very close to sensitivities of other sensors in the group at any given time of the sensor session. While not wishing to be bound by theory, it is believed that the observed upward trend in sensitivity (over time), which is particularly pronounced in the early part of the sensor session, can be attributed to conditioning and hydration of sensing regions of the working electrode. It is also believed that the glucose concentration of the fluid surrounding the continuous glucose sensor during startup of the sensor can also affect the sensitivity drift.

With the sensors tested in the example study illustrated by FIG. 13, the change in sensor sensitivity (expressed as a percentage of a substantially steady state sensitivity), over a time defined by a sensor session, resembled a logarithmic growth curve. It should be understood that other continuous analyte sensors fabricated with different techniques, with different specifications (e.g., different membrane thickness or composition), or under different manufacturing conditions, may exhibit a different sensor sensitivity profile (e.g., one associated with a linear function). Nonetheless, with improved control over operating conditions of the sensor fabrication process, high levels of reproducibility may be achieved, such that sensitivity profiles exhibited by individual sensors of a sensor population (e.g., a sensor lot) are substantially similar and sometimes nearly identical.

In various examples, the change or drift in sensitivity over a sensor session is not only substantially consistent among sensors manufactured in substantially the same way under substantially same conditions, but also that modeling can be performed through mathematical functions that can accurately estimate this change or drift. As illustrated in FIG. 13, an estimative algorithm function 1320 can be used to define the relationship between time during the sensor session and sensor sensitivity. The estimative algorithm function may be generated by testing a sample set (comprising one or more sensors) from a sensor lot under in vivo and/or in vitro conditions. Alternatively, the estimative algorithm function may be generated by testing each sensor under in vivo and/or in vitro conditions.

In some examples, a sensor may undergo an in vitro sensor sensitivity drift test, in which the sensor is exposed to changing conditions (e.g., step changes of glucose concentrations in a solution), and an in vitro sensitivity profile of the sensor is generated over a certain time period. The time period of the test may substantially match an entire sensor session of a corresponding in vivo sensor, or it may encompass a portion of the sensor session (e.g., the first day, the first two days, or the first three days of the sensor session, etc.). It is contemplated that the above-described test may be performed on each individual sensor, or alternatively on one or more sample sensors of a sensor lot. From this test, an in vitro sensitivity profile may be created, from which an in vivo sensitivity profile may be modeled and/or formed.

From the in vivo or in vitro testing, one or more data sets, each comprising data points associating sensitivity with time, may be generated and plotted. A sensitivity profile or curve can then be fitted to the data points. If the curve fit is determined to be satisfactory (e.g., if the standard deviation of the generated data points is less a certain threshold), then the sensor sensitivity profile or curve may be judged to have passed a quality control and be suitable for release. From there, the sensor sensitivity profile can be transformed into an estimative algorithm function or alternatively into a look-up table. The algorithm function or look-up table can be stored in a computer-readable memory, for example, and accessed by a computer processor.

The estimative algorithm function may be formed by applying curve fitting techniques that regressively fit a curve to data points by adjusting the function (e.g., by adjusting constants of the function) until an optimal fit to the available data points is obtained. For example, a “curve” (i.e., a function sometimes referred to as a “model”) may be fitted and generated that relates one data value to one or more other data values and selecting parameters of the curve such that the curve estimates the relationship between the data values. By way of example, selection of the parameters of the curve may involve selection of coefficients of a polynomial function. In some examples, the curve fitting process may involve evaluating how closely the curve determined in the curve fitting process estimates the relationship between the data values, to determine the optimal fit. The term “curve,” as used herein, is a broad term, and is to be given its ordinary and customary meaning to a person of ordinary skill in the art (and is not to be limited to a special or customized meaning), and refers to a function or a graph of a function, which can involve a rounded curve or a straight curve, i.e., a line.

The curve may be formed by any of a variety of curve fitting techniques, such as, for example, the linear least squares fitting method, the non-linear least squares fitting method, the Nelder-Mead Simplex method, the Levenberg-Marquardt method, and variations thereof. In addition, the curve may be fitted using any of a variety of functions, including, but not limited to, a linear function (including a constant function), logarithmic function, quadratic function, cubic function, square root function, power function, polynomial function, rational function, exponential function, sinusoidal function, and variations and combinations thereof. For example, in some examples, the estimative algorithm comprises a linear function component which is accorded a first weight w1, a logarithmic function component which is accorded a second weight w2, and an exponential function component which is accorded a third weight w3. In further examples, the weights associated with each component can vary as a function of time and/or other parameters, but in alternative example, one or more of these weights are constant as a function of time.

In various examples, the estimative algorithm function's correlation (e.g., R2 value), which is a measure of the quality of the fit of the curve to the data points, with respect to data obtained from the sample sensors, may be one metric used to determine whether a function is optimal. In various examples, the estimative algorithm function formed from the curve fitting analysis may be adjusted to account for other parameters, e.g., other parameters that may affect sensor sensitivity or provide additional information about sensor sensitivity. For example, the estimative algorithm function may be adjusted to account for the sensitivity of the sensor to hydrogen peroxide or other chemical species.

Estimative algorithms formed and used to accurately estimate an individual sensor's sensitivity, at any time during a sensor session, can be based on factory calibration and/or based on a single early reference measurement (e.g., using a single point blood glucose monitor). In some examples, sensors across a population of continuous analyte sensors manufactured in substantially the same way under substantially same conditions exhibit a substantially fixed in vivo to in vitro sensitivity relationship. For example, in one example, the in vivo sensitivity of a sensor at a certain time after start of sensor use (e.g., at t=about 5, 10, 15, 30, 60, 120, or 180 minutes after sensor use) is consistently equal to a measured in vitro sensitivity of the sensor or of an equivalent sensor. From this relationship, an initial value of in vivo sensitivity can be generated, from which an algorithmic function corresponding to the sensor sensitivity profile can be formed. In some examples, from this initial value (which represents one point in the sensor sensitivity profile), the rest of the sensor sensitivity profile can be determined and plotted. The initial value of in vivo sensitivity can be associated with any portion of the sensor sensitivity profile. In various examples, multiple initial values of in vivo sensitivities, which are time-spaced apart, and which correspond to multiple in vitro sensitivities, can be calculated and combined together to generate the sensor sensitivity profile.

With some examples, it has been found that not only does the sensor's sensitivity tend to drift over time, but that the sensor's baseline also drifts over time. Accordingly, in various examples, the concepts behind the methods and systems used to predict sensitivity drift can also be applied to create a model that predicts baseline drift over time. Although not wishing to be bound by theory, it is believed that the total signal received by the sensor electrode is comprised of a glucose signal component, an interference signal component, and an electrode-related baseline signal component that is a function of the electrode and that is substantially independent of the environment (e.g., extracellular matrix) surrounding the electrode. As noted above, the term “baseline,” as used herein, refers without limitation to the component of an analyte sensor signal that is not related to the analyte concentration. Accordingly, the baseline, as the term is defined herein, is comprised of the interference signal component and the electrode-related baseline signal component. Again, while not wishing to be bound by theory, it is believed that increased membrane permeability typically not only results in an increased rate of glucose diffusion across the sensor membrane, but also in an increased rate of diffusion of interferents across the sensor membrane. Accordingly, changes in sensor membrane permeability over time, which causes sensor sensitivity drift, will similarly also likely cause the interference signal component of the baseline to drift. Simply put, the interference signal component of the baseline is not static, and is typically changing as a function of time, which, in turn, causes the baseline to also drift over time. By analyzing how each of the aforementioned components of the baseline reacts to changing conditions and to time (e.g., as a function of time, temperature), a predictive model can be developed to predict how the baseline of a sensor will drift during a sensor session. By being able to prospectively predict both sensitivity and baseline of the sensor, it is believed that a factory calibrated or automatically self-calibrating continuous analyte sensor can be achieved, i.e., a sensor that does not require use of reference measurements (e.g., a fingerstick measurement) for calibration.

### Calibration Code

The process of manufacturing continuous analyte sensors may sometimes be subjected to a degree of variability between sensor lots, as will be described in greater detail below. To compensate for this variability, one or more calibration codes may be assigned to each sensor or sensor set to define parameters that can affect sensor sensitivity or provide additional information about the sensitivity profile. The calibration codes can reduce variability in the different sensors, ensuring that the results obtained from using sensors from different sensors lots will be generally equal and consistent by applying an algorithm that adjusts for the differences. In one example, the analyte sensor system may be configured such that one or more calibration codes are to be manually entered into the system by a user. In other examples, the calibration codes may be part of a calibration encoded label that is adhered to (or inserted into) a package of multiple sensors. The calibration encoded label itself may be read or interrogated by any of a variety of techniques, including, but not limited to, optical techniques, RFID (radio-frequency identification), or the like, and combinations thereof. These techniques for transferring the code to the sensor system may be more automatic, accurate, and convenient for the patient, and less prone to error, as compared to manual entry. Manual entry, for instance, possesses the inherent risk of an error caused by a patient or hospital staff entering the wrong code, which can lead to an incorrect calibration, and thus inaccurate glucose concentration readings. In turn, this may result in a patient or hospital staff taking an inappropriate action (e.g., injecting insulin while in a hypoglycemic state).

In some examples, calibration codes assigned to a sensor may include a first calibration code associated with a predetermined logarithmic function corresponding to a sensitivity profile, a second calibration code associated with an initial in vivo sensitivity value, and other calibration codes, with each code defining a parameter that affects sensor sensitivity or provides information about sensor sensitivity. The other calibration codes may be associated with any a priori information or parameter described elsewhere herein and/or any parameter that helps define a mathematical relationship between the measured signal and analyte concentration. The calibration code may be developed from these measurements or may be developed based on manufacturing parameters known, determined, or measured during fabrication of, e.g., a lot, or by a combination of these. In certain embodiments, parameters and/or values associated with calibration of a sensor (e.g., one or more sensitivity values, one or more baseline values, etc.) may be stored directly a memory associated with the sensor (e.g., in sensor electronics operably connected with the sensor). The storage of the parameters and/or values in the memory may be stored during sensor manufacture (e.g., at the factory) and/or may be stored in the memory at some part subsequent to manufacture (e.g., after the sensor has been inserted within a body of a patient).

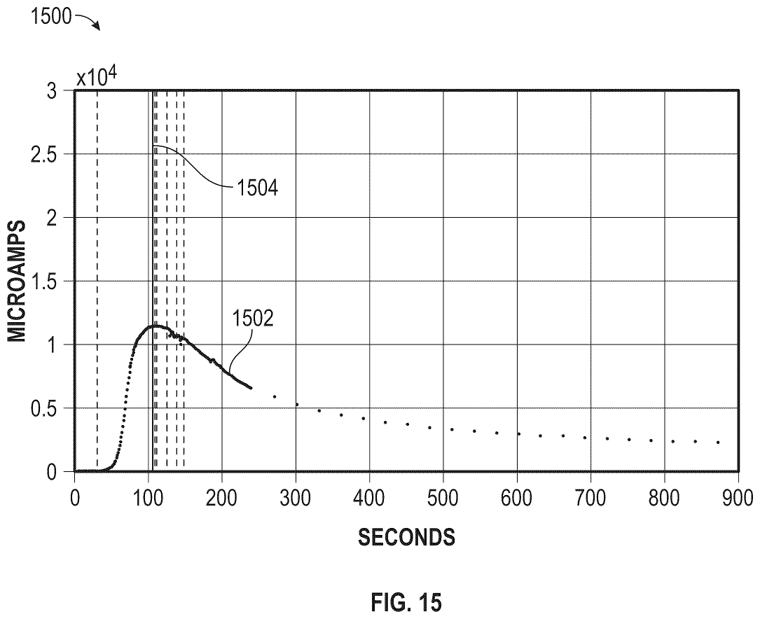

FIG. 15 is a diagram showing one example of a plot 1500 of a sensor signal 1502 generated by an example analyte (e.g., glucose) sensor after insertion into a host. The plot 1500 includes a horizontal axis indicating time in seconds and a vertical axis indicating the magnitude of the sensor signal 1502. In this example, the magnitude of the sensor signal 1502 is indicated as a current and measured in microamps.

The zero time on the horizontal axis corresponds to the time at which the analyte sensor was inserted into the host. In this example, the magnitude of the sensor signal 1502 remains near to zero for about 40 seconds and then begins to rise. Shortly after 100 seconds, the magnitude of the sensor signal 1502 peaks and begins to decline. The time of peak of the sensor signal 1502 is indicated by line 1504. It will be appreciated that the plot 1500 shown in FIG. 15 is just one example of the behavior of an analyte sensor after insertion into a host. It will be appreciated that different analyte sensors may generate sensor signals having different properties. For example, as described herein, sensors subject to different sterilization techniques may exhibit post-insertion sensor signals having different properties.

In various examples, one or more properties of the post-insertion sensor signal 1502 may affect the sensitivity of the analyte sensor during a sensor session. One example property of the sensor signal 1502 is a time-to-peak. The time-to-peak is a measure of an elapsed time between the insertion of the analyte sensor into the host and the peak of the sensor signal generated by the analyte sensor. In the example of FIG. 15, the time-to-peak is a little over 100 seconds. Other example properties of the sensor signal 1502 may include a time from sensor insertion until the sensor signal begins to rise, a slope of the sensor signal as it approaches the peak, a magnitude of the sensor signal 1502 at the peak, and/or any other suitable properties of the sensor signal.

In various examples, one or more properties of the post-insertion sensor signal 1502 may be used to generate (e.g., calculate and/or modify) a sensitivity or sensitivity trend for the analyte sensor. The sensitivity may be used to generate estimated analyte concentrations from the sensor signal 1502, as described herein. The sensitivity determined based on the post-insertion sensor signal 1502 may be used during break-in for the sensor. After break-in, a predetermined sensitivity or sensitivity model may be used, for example, as described herein. In some examples, a sensitivity determined based on the post-insertion sensor signal properties may be used throughout a sensor session for the analyte sensor.

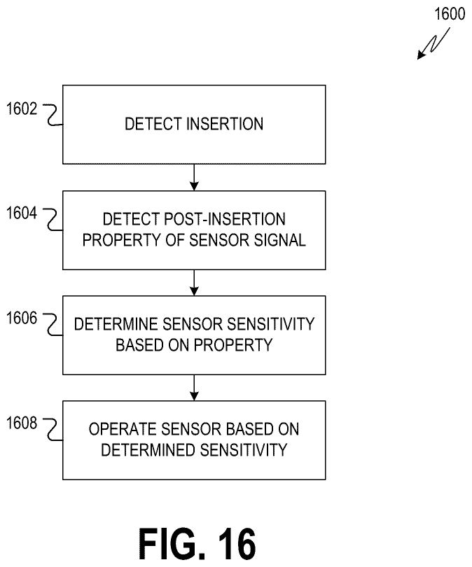

FIG. 16 is a flowchart showing one example of a process flow 1600 that may be executed in an analyte sensor system, such as for example, the analyte sensor system 102 described herein, to determine a sensitivity of the analyte sensor based on a post-insertion sensor signal. In some examples, the process flow 1600 is performed by sensor electronics of an analyte sensor system, such as the sensor electronics 106 of the analyte sensor system 102 described herein.

At operation 1602, the sensor electronics may detect insertion of the analyte sensor into a host. The detection may be performed in any suitable manner. In some examples, the sensor electronics may detect insertion by detecting the presence of a sensor signal generated by the analyte sensor. The presence of a sensor signal may indicate that the analyte sensor is in contact with a bodily fluid of the host and reacting with the bodily fluid to generate a sensor signal. In some examples, the sensor electronics may detect insertion of the analyte sensor based on a signal received from a deployment sensor, such as the deployment sensor 1210, the transmitters 985 and 1112, or another suitable sensor that generates a signal when the analyte sensor is inserted into a host. In some examples, the sensor electronics may detect insertion of the analyte sensor into a host based on detecting the sensor signal in conjunction with receiving a signal from an insertion sensor. In some examples, the sensor electronics may detect insertion of the analyte sensor into a host based on user input and/or via communication signal from a receiver device (e.g., a smart phone).

At operation 1604, the sensor electronics may detect at least one post-insertion property of the sensor signal. The at least one post-insertion property may include, for example, a time-to-peak of the sensor signal, a time until the sensor signal begins to rise, a slope of the sensor signal, a magnitude of the sensor signal at the peak, and/or any other suitable post-insertion property of the sensor signal.

At operation 1606, the sensor electronics may determine a sensor sensitivity based on the property of the post-insertion sensor signal. In some examples, the sensitivity may be based on a sensitivity or sensitivity model for the analyte sensor him determined without regard to the post-insertion sensor signal, such as a sensitivity or sensitivity model indicated by a calibration code and/or calibration properties stored in memory as described herein. In some examples, this may include adding one or more terms to an existing expression of the sensor sensitivity where the one or more added terms are based on the time-to-peak. Consider Equation [2] below, which shows an example expression for a sensor sensitivity mₒ that is based on a previous calibration, such as, for example, a factory calibration or other suitable calibration:

$${m}_{o} = a + b cal \qquad [ 2 ]$$

In Equation [2], a is a scalar and b is a scalar coefficient of a calibration term cal. The scalars a and b, as well as the calibration term cal may be based on a factory calibration or other suitable calibration of the analyte sensor, for example, as described herein.

Equation [3] shows an example expression of a sensor sensitivity. Relative to Equation [2], Equation [3] includes an additional term with a scalar c that is a coefficient of an additional term pip. The term pip is a term of that either is or is based on the value of at least one post-insertion property of the sensor signal, such as time-to-peak.

$${m}_{o} = a + b cal + c pip \qquad [ 3 ]$$

In the example of Equation [3], the sensitivity mₒ is an additive combination of the scalar “a,” the term based on the calibration of the analyte sensor “b cal,” and the term based on the post-insertion property, “c pip.”

Equation [4] shows another example expression of a sensitivity that may be determined based on at least one post-insertion property of the sensor signal. The example of Equation [4], the sensitivity mₒ is an additive combination of the scalar “a,” the term based on the calibration of the analyte sensor “b cal,” and a term based on the post-insertion property and the calibration, “c cal pip.”

$${m}_{o} = a + b cal + c cal pip \qquad [ 4 ]$$

Equation [5] shows another example expression of a sensitivity that may be determined based on at least one post-insertion property of the sensor signal. The example of Equation [5], the sensitivity mₒ is an additive combination of the scalar “a,” the term based on the calibration of the analyte sensor “b cal,” a term based on the post-insertion property “c pip,” and a term based on the post-insertion property and the calibration, “d cal pip.”

$${m}_{o} = a + b cal + c pip + d pip cal \qquad [ 5 ]$$

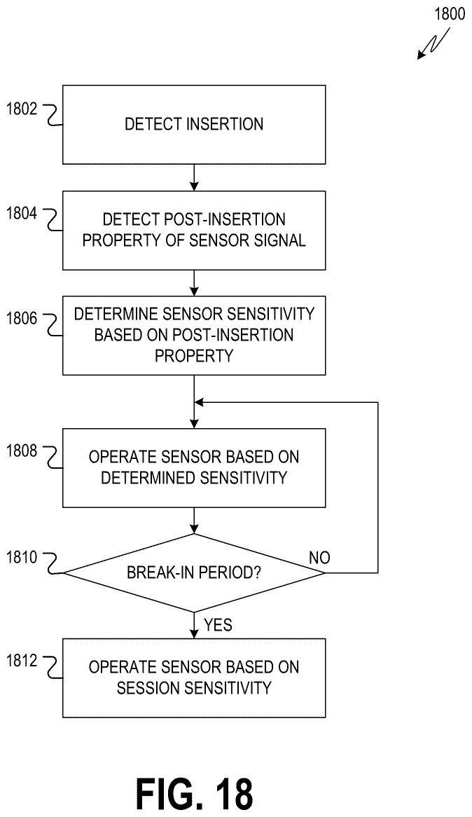

In some examples, the sensor sensitivity determined based on the post-insertion property at operation 1606 may be a break-in sensitivity used during a break-in of the analyte sensor. For example, at the conclusion of the break-in, the sensor electronics may cease to use the break-in sensitivity and may begin using a session sensitivity for (at least a portion of) the remainder of the sensor session. An example process flow 1800 utilizing a break-in sensitivity and a session sensitivity is described herein with respect to FIG. 18.

At operation 1608, the sensor electronics may operate the analyte sensor system using the sensitivity determined at operation 1606. For example, the sensor electronics may receive sensor signal data and apply the sensitivity to the received sensor signal data to generate one or more estimated analyte concentrations for the host.

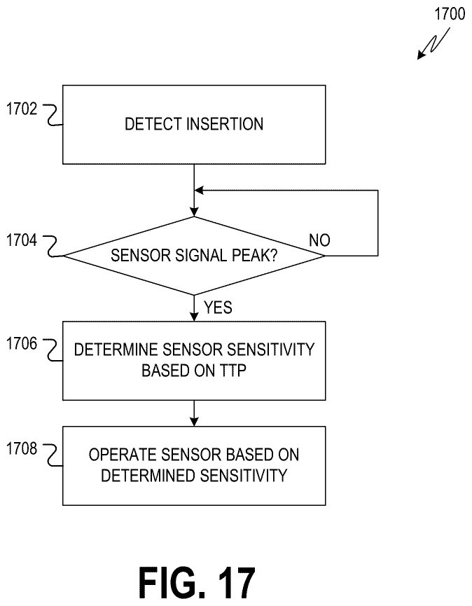

FIG. 17 is a flowchart showing another example of a process flow 1700 that may be executed in an analyte sensor system, such as, for example, the analyte sensor system 102 described herein, to determine a sensitivity of the analyte sensor based on a post-insertion sensor signal. The process flow 1700 determines a sensitivity for the analyte sensor using a time-to-peak, as described herein.

At operation 1702, the sensor electronics detects insertion of the analyte sensor, for example, as described herein with respect to operation 1602. At operation 1704, the sensor electronics determine if the sensor signal generated by the analyte sensor has reached a peak. The sensor electronics may determine whether the sensor signal has reached a peak in any suitable manner. In some examples, the sensor electronics may monitor a slope of the sensor signal. If the slope of the sensor signal transitions from positive to negative, the sensor electronics may detect a peak. If no sensor signal peak is detected at operation 1704, the sensor electronics may continue monitoring the sensor signal to detect the peak. In some examples, the sensor electronics may perform checks to distinguish one or more local peaks from an absolute peak.

When a sensor signal peak is detected, the sensor electronics may, at operation 1706, determine a sensor sensitivity based on the time-to-peak. The sensor electronics may determine the sensor sensitivity in any suitable manner. In some examples, the sensor electronics may determine a sensor sensitivity based on one or more of the Equations [2]-[5] described herein. In some examples, the sensor sensitivity determined at operation 1706 is a break-in sensitivity that is used during a break-in for the analyte sensor, with a session sensitivity used thereafter. Also, in some examples, the sensor sensitivity determined that operation 1706 is used beyond the break-in including, for example, at later portions of the sensor session.

At operation 1708, the sensor electronics may operate the analyte sensor system using the sensitivity determined at operation 1606. For example, the sensor electronics may receive sensor signal data and apply the sensitivity to the received sensor signal data to generate one or more estimated analyte concentrations for the host.

FIG. 18 is a flowchart showing another example of a process flow 1800 that may be executed in an analyte sensor system, such as for example, the analyte sensor system 102 described herein, to determine a sensitivity of the analyte sensor based on a post-insertion sensor signal. In the example of FIG. 18, a break-in sensitivity is determined using a post-insertion property of the sensor signal. The break-in sensitivity is used during the break-in of the analyte sensor. After the break-in of the analyte sensor, the sensor electronics use a session sensitivity.

At operation 1802, the sensor electronics detects insertion of the analyte sensor, for example, as described herein with respect to operation 1602. At operation 1804, the sensor electronics may detect at least one post-insertion property of the sensor signal. The at least one post-insertion property may include, for example, a time-to-peak of the sensor signal, a time until the sensor signal begins to rise, a slope of the sensor signal, a magnitude of the sensor signal at the peak, and/or any other suitable post-insertion property of the sensor signal.

At operation 1806, the sensor electronics determine a sensor sensitivity based on a post-insertion property, such as the time-to-peak. The sensor electronics may determine the sensor sensitivity in any suitable manner. In some examples, the sensor electronics may determine a sensor sensitivity based on one or more of the Equations [2]-[5] described herein. At operation 1808, the sensor electronics may operate the analyte sensor system using the sensitivity determined at operation 1806. For example, the sensor electronics may receive sensor signal data and apply the sensitivity to the received sensor signal data to generate one or more estimated analyte concentrations for the host. In this example, the sensor sensitivity determined at operation 1806 may be a break-in sensitivity to be used during a break-in of the analyte sensor.

At operation 1810, the sensor electronics may determine if the break-in for the analyte sensor is concluded. The sensor electronics may determine that the break-in is concluded in any suitable manner. In some examples, the sensor electronics may determine that the break-in is concluded if more than a threshold time has passed since the insertion of the analyte sensor. The threshold time may be any suitable time such as, for example, 30 minutes, 60 minutes, 90 minutes, 120 minutes, 150 minutes, and/or the like. In some examples, the sensor electronics may detect that break-in has concluded based on the sensor signal generated by the analyte sensor. For example, after break-in, the sensor signal generated by the analyte sensor may be substantially linear. The sensor electronics may monitor the sensor signal to determine its linearity. When the sensor signal demonstrates a linearity exceeding a linearity threshold, the sensor electronics may determine that the break-in has concluded.

If the sensor electronics determines at operation 1810 that the break-in for the analyte sensor has not concluded, then it may return to operation 1808 and continue operating the analyte sensor based on the break-in sensitivity determined at operation 1806. If the sensor electronics determines at operation 1810 that the break-in for the analyte sensor has concluded, then it may, at operation 1812, operate the sensor based on a session sensitivity that is different than the break-in sensitivity. For example, referring to Equations [2]-[5] above, a session sensitivity may be derived from a break-in sensitivity by omitting terms of the break-in sensitivity that are based on a post-insertion property such as time-to-peak.

In some examples, an analyte sensor system may be configured to sample the sensor signal at a higher frequency after insertion and a lower frequency later in the sensor session. For example, higher frequency sampling of the sensor signal after insertion may allow the sensor electronics to more accurately measure post-insertion properties of the sensor signal and thereby more accurately determine the sensor sensitivity based on the post-insertion properties.

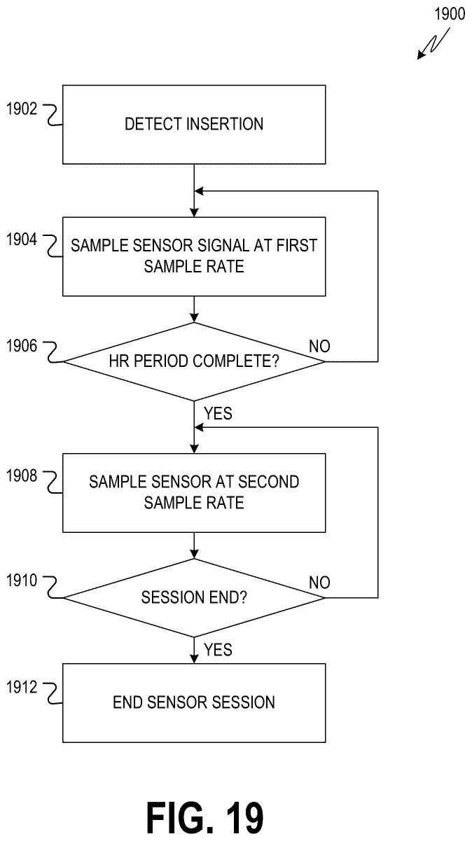

FIG. 19 is a flowchart showing another example of a process flow 1900 that may be executed in an analyte sensor system, such as, for example, the analyte sensor system 102 described herein, to measure a sensor signal after insertion. At operation 1902, the sensor electronics detects insertion of the analyte sensor, for example, as described herein with respect to operation 1602. At operation 1904, the sensor electronics may begin sampling the sensor signal at a first sample rate. The first sample rate may be a relatively high sample rate such as, for example, three samples per second, two samples per second, one sample per second, one and one half samples per second, one sample every two seconds, and/or the like.

The sensor electronics may continue to sample the sensor signal at the first sample rate during a high-resolution sampling period. At operation 1906, the sensor electronics may determine if the high-resolution sampling period is complete. The high-resolution sampling period may last for any suitable amount of time. In some examples, the high-resolution sampling period may last for a predetermined amount of time after insertion of the analyte sensor such as, for example, 30 seconds after insertion, 60 seconds after insertion, 90 seconds after insertion, 120 seconds after insertion, 150 seconds after insertion, and/or the like. In some examples, the high-resolution sampling period may extend through the break-in of the analyte sensor. For example, the sensor electronics may detect the end of the break-in for the analyte sensor, for example, as described herein with respect to operation 1810. The conclusion of the break-in may also be the conclusion of the high-resolution sampling period.

If the high-resolution sampling period is not complete at operation 1906, the sensor electronics may continue to sample the sensor signal at the first sample rate at operation 1904. If the high-resolution sampling period is complete at operation 1906, then the sensor electronics may sample the sensor at a second sample rate at operation 1908. The second sample rate may be lower than the first sample rate. For example, the second sensor rate may be one sample per 10 seconds, one sample per 15 seconds, one sample per 20 seconds, one sample per 30 seconds, one sample per 40 seconds, one sample per 50 seconds, one sample per 60 seconds, one sample per 70 seconds, and/or the like.

At operation 1910, the sensor electronics may determine if the sensor session for the analyte sensor has ended. The sensor session for the analyte sensor may end, for example, after a predetermined time has elapsed since insertion (e.g., 10 days, 11 days, 12 days, 13 days, 14 days, 15 days, 16 days, and/or the like). In some examples, the sensor session for the analyte sensor may also end if there is detected an error in the analyte sensor system. If the sensor electronics does not determine that the sensor session has ended at operation 1910, then the sensor electronics may continue to sample the sensor signal at the second sample rate at operation 1908. If the sensor electronics does determine that the sensor session has ended at operation 1910, then the sensor electronics may end the sensor session at operation 1912. Ending the sensor session may include, for example, ceasing to provide estimated analyte concentrations to the host or other user, ceasing to provide a biased signal to the analyte sensor, sending an alert message to the host or other user indicating the end of the sensor session, and/or the like.

In some examples, the process flow 1900 may be executed concurrent with one or more of the process flows 1600, 1700, and/or 1800. For example, the post-insertion properties of the sensor signal may be taken during the high-resolution period. In some examples, this may permit the detection of post-insertion properties that may not be measurable at the lower sample rate used after the high-resolution period.

In some examples, a contribution to sensor signal due to factors present during break-in may be modeled and accounted for during break-in. Consider Equation [6] below:

$$x = ( {y}_{analyte} - {y}_{BreakIn} ) / m - b \qquad [ 6 ]$$

Equation [6] represents a solution to Equation [1] above in which x represents the estimated glucose concentration (mg/dL), m represents the sensor sensitivity to the analyte (counts/mg/dL), and b represents the baseline or compartment signal. In this example, the total sensor signal y during breaking is the difference between an analyte signal portion resulting from the presence of analyte at the sensor (y_{analyte}) and a break-in signal portion caused by factors present at break-in (y_{BreakIn}). In some examples, the analyte (e.g., glucose) concentration may be determined during break-in utilizing the relationships described by Equation [6].

In some examples, the break-in signal portion may be modeled as having a component due to break-in and a model due to hydration, for example, as illustrated by Equation [7] below:

$${y}_{BreakIn} = f ( breakIn ( t ) , hydration ( t ) ) \qquad [ 7 ]$$

In this example, breakIn(t) is a function of time that models changes in sensor signal due to ion and corresponding currents generated at the sensor during break-in. The value of breakIn(t) may initially be large and may decay as break-in proceeds. The function hydration(t) is also a function of time and models hydration behavior at the sensor, for example, related to the transportation of ions at the sensor. For example, as the sensor becomes hydrated, ion transport may increase, causing a corresponding increase in current or sensor signal. The function hydration(t) may be an increasing function that starts at 0% at insertion and increases to 100%.

In some examples, the functions, breakIn(t) and hydration(t) may be modeled by fitting a suitable distribution to observed data. Any suitable distribution may be used to model breakIn(t) and hydration(t). For example, breakIn(t) may be modeled using any suitable distribution that is initially large and the case as time increases. An example of such a distribution is given by Equation [8] below:

$$( {a}_{0} + {a}_{1} * {e}^{- {λ}_{1} t} + {a}_{2} * {e}^{- {λ}_{2} t} + {a}_{3} * {t}^{- {λ}_{3}} ) \qquad [ 8 ]$$

Similarly, the hydration(t) may be modeled using any suitable distribution that increases over time from 0% to 100%. Examples of such distributions include cumulative density functions such as CDF, normal distributions, logistic distributions, exponential distributions, log-logistic distributions, and/or the like. An example distribution that may be used to model hydration(t) is given by Equation [9] below:

$$\frac{1}{1 + {( \frac{time}{α} )}^{- β}} \qquad [ 9 ]$$

An expression of the break-in signal portion y_{BreakIn} utilizing the example distribution of Equation [8] for breakIn(t) and the example distribution of Equation [9] for hydration(t) is given by Equation below:

$${y}_{BreakIn} = ( {a}_{0} + {a}_{1} * {e}^{- {λ}_{1} t} + {a}_{2} * {e}^{- {λ}_{2} t} + {a}_{3} * {t}^{- {λ}_{3}} ) * 1 - \frac{1}{1 + {( \frac{time}{α} )}^{- β}}$$

[10]. In the example of Equation [10], coefficients for the utilized distributions include (α₀, α₁, α₂, α₃, α, λ₁, λ₂, λ₃, β).

In some examples, values for the coefficients used to model the break-in signal portion y_{BreakIn} may be determined utilizing observed data. For example, data may be gathered utilizing a non-enzyme sensor. A non-enzyme sensor is an analyte sensor that does not include the enzyme described herein that catalyzes a reaction with glucose or another analyte. Accordingly, a non-analyte sensor may not generate an analyte signal portion (y_{analyte} from Equation [6] above). Accordingly, a non-enzyme sensor, during break-in, may generate a break-in signal portion y_{BreakIn} only. The break-in signal generated by a non-enzyme sensor, or a number of non-enzyme sensors, may be compared to a break-in signal portion model, such as the model given by Equation above to derive values for the coefficients of the break-in signal portion model. The resulting break-in signal portion model may be utilized during break-in for other analyte sensors.

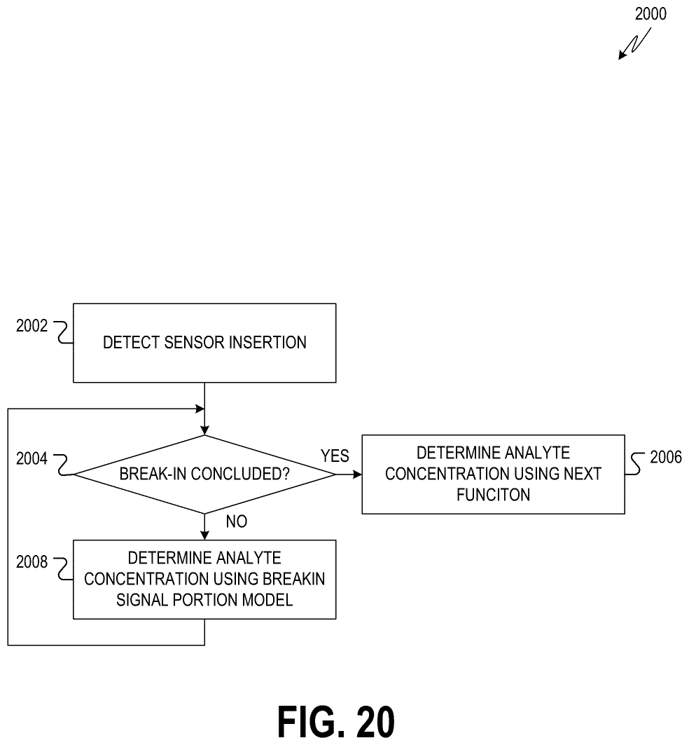

FIG. 20 is a flowchart showing one example of a process flow 2000 that may be executed by an analyte sensor (e.g., sensor electronics thereof) to operate the analyte sensor during break-in utilizing a break-in signal portion model. At operation 2002, the sensor electronics may detect sensor insertion, for example, as described herein. At operation 2004, the sensor electronics may determine if the break-in for the analyte sensor is concluded. The sensor electronics may determine that the break-in is concluded in any suitable manner. In some examples, the sensor electronics may determine that the break-in is concluded if more than a threshold time has passed since the insertion of the analyte sensor. The threshold time may be any suitable time such as, for example, 1 minute, 3 minutes, 5 minutes, 30 minutes, 60 minutes, 90 minutes, 120 minutes, 150 minutes, and/or the like. In some examples, the sensor electronics may detect that break-in has concluded based on the sensor signal generated by the analyte sensor. For example, after break-in, the sensor signal generated by the analyte sensor may be substantially linear. The sensor electronics may monitor the sensor signal to determine its linearity. When the sensor signal demonstrates a linearity exceeding a linearity threshold, the sensor electronics may determine that the break-in has concluded.

If the sensor break-in has not concluded, then the sensor electronics may, at operation 2006, determine an analyte concentration using a sensor signal and a model considering a break-in signal portion, for example, as described herein with respect to Equation [6]. If the sensor break-in has concluded, the sensor electronics may, at operation 2008, determine an analyte concentration using a model that does not consider break-in signal portion, such as, for example, Equation [1] described herein.

In some examples, different models for the analyte sensor break-in period may be used together. For example, a model considering a break-in signal portion, such as described herein with respect to Equation [6] may be used in conjunction with a model that utilizes a modified sensor sensitivity during break-in.

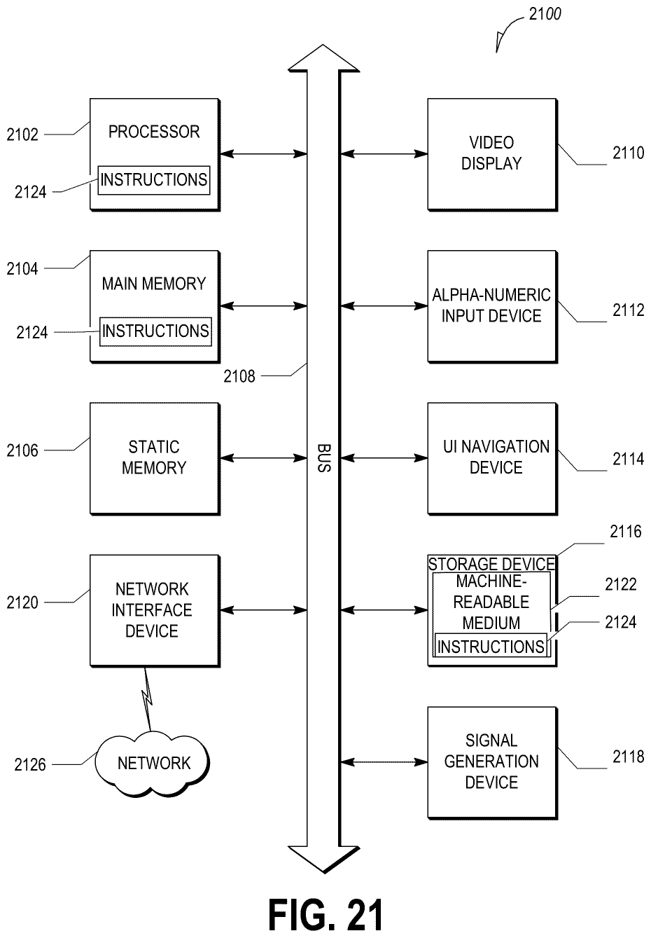

FIG. 21 is a block diagram illustrating a computing device hardware architecture 2100, within which a set or sequence of instructions can be executed to cause a machine to perform examples of any one of the methodologies discussed herein. The hardware architecture 2100 can describe various computing devices including, for example, the sensor electronics 106, the peripheral medical device 122, the smart device 112, the tablet 114, etc.

The architecture 2100 may operate as a standalone device or may be connected (e.g., networked) to other machines. In a networked deployment, the architecture 2100 may operate in the capacity of either a server or a client machine in server-client network environments, or it may act as a peer machine in peer-to-peer (or distributed) network environments. The architecture 2100 can be implemented in a personal computer (PC), a tablet PC, a hybrid tablet, a set-top box (STB), a personal digital assistant (PDA), a mobile telephone, a web appliance, a network router, a network switch, a network bridge, or any machine capable of executing instructions (sequential or otherwise) that specify operations to be taken by that machine.

The example architecture 2100 includes a processor unit 2102 comprising at least one processor (e.g., a central processing unit (CPU), a graphics processing unit (GPU), or both, processor cores, compute nodes). The architecture 2100 may further comprise a main memory 2104 and a static memory 2106, which communicate with each other via a link 2108 (e.g., bus). The architecture 2100 can further include a video display unit 2110, an input device 2112 (e.g., a keyboard), and a UI navigation device 2114 (e.g., a mouse). In some examples, the video display unit 2110, input device 2112, and UI navigation device 2114 are incorporated into a touchscreen display. The architecture 2100 may additionally include a storage device 2116 (e.g., a drive unit), a signal generation device 2118 (e.g., a speaker), a network interface device 2120, and one or more sensors (not shown), such as a Global Positioning System (GPS) sensor, compass, accelerometer, or another sensor.

In some examples, the processor unit 2102 or another suitable hardware component may support a hardware interrupt. In response to a hardware interrupt, the processor unit 2102 may pause its processing and execute an ISR, for example, as described herein.

The storage device 2116 includes a machine-readable medium 2122 on which is stored one or more sets of data structures and instructions 2124 (e.g., software) embodying or used by any one or more of the methodologies or functions described herein. The instructions 2124 can also reside, completely or at least partially, within the main memory 2104, within the static memory 2106, and/or within the processor unit 2102 during execution thereof by the architecture 2100, with the main memory 2104, the static memory 2106, and the processor unit 2102 also constituting machine-readable media.

### Executable Instructions and Machine-Storage Medium

The various memories (i.e., 2104, 2106, and/or memory of the processor unit(s) 2102) and/or storage device 2116 may store one or more sets of instructions and data structures (e.g., instructions) 2124 embodying or used by any one or more of the methodologies or functions described herein. These instructions, when executed by processor unit(s) 2102 cause various operations to implement the disclosed examples.

As used herein, the terms “machine-storage medium,” “device-storage medium,” “computer-storage medium” (referred to collectively as “machine-storage medium 2122”) mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 2122 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage media, computer-storage media, and device-storage media 2122 specifically exclude carrier waves, modulated data signals, and other such media, at least some of which are covered under the term “signal medium” discussed below.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer-Readable Medium

The terms “machine-readable medium,” “computer-readable medium” and “device-readable medium” mean the same thing and may be used interchangeably in this disclosure. The terms are defined to include both machine-storage media and signal media. Thus, the terms include both storage devices/media and carrier waves/modulated data signals.

The instructions 2124 can further be transmitted or received over a communications network 2126 using a transmission medium via the network interface device 2120 using any one of a number of well-known transfer protocols (e.g., HTTP). Examples of communication networks include a LAN, a WAN, the Internet, mobile telephone networks, plain old telephone service (POTS) networks, and wireless data networks (e.g., Wi-Fi, 3G, 4G LTE/LTE-A, 5G or WiMAX networks). The term “transmission medium” shall be taken to include any intangible medium that is capable of storing, encoding, or carrying instructions for execution by the machine, and includes digital or analog communications signals or other intangible media to facilitate communication of such software.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Various components are described in the present disclosure as being configured in a particular way. A component may be configured in any suitable manner. For example, a component that is or that includes a computing device may be configured with suitable software instructions that program the computing device. A component may also be configured by virtue of its hardware arrangement or in any other suitable manner.

The above description is intended to be illustrative, and not restrictive. For example, the above-described examples (or one or more aspects thereof) can be used in combination with others. Other examples can be used, such as by one of ordinary skill in the art upon reviewing the above description. The Abstract is to allow the reader to quickly ascertain the nature of the technical disclosure, for example, to comply with 37 C.F.R. § 1.72(b) in the United States of America. It is submitted with the understanding that it will not be used to interpret or limit the scope or meaning of the claims.

Also, in the above Detailed Description, various features can be grouped together to streamline the disclosure. However, the claims cannot set forth every feature disclosed herein, as examples can feature a subset of said features. Further, examples can include fewer features than those disclosed in a particular example. Thus, the following claims are hereby incorporated into the Detailed Description, with each claim standing on its own as a separate example. The scope of the examples disclosed herein is to be determined with reference to the appended claims, along with the full scope of equivalents to which such claims are entitled.

Each of these non-limiting examples in any portion of the above description may stand on its own or may be combined in various permutations or combinations with one or more of the other examples.

The above detailed description includes references to the accompanying drawings, which form a part of the detailed description. The drawings show, by way of illustration, specific embodiments in which the subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” “third,” etc., are used merely as labels, and are not intended to impose numerical requirements on their objects.

Geometric terms, such as “parallel”, “perpendicular”, “round”, or “square” are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the subject matter should be determined with reference to the claims, along with the full scope of equivalents to which such claims are entitled.

What is claimed is:

1. An analyte sensor system for in vivo use, the analyte sensor system comprising:
an analyte sensor; and
sensor electronics in electrical communication with the analyte sensor, the sensor electronics being configured to perform operations comprising:
accessing an indication that the analyte sensor has been inserted into a host;
detecting a property of a sensor signal generated by the analyte sensor after being inserted into the host;
determining a first sensor sensitivity based at least in part on the property of the sensor signal; and
determining a first analyte concentration using the sensor signal and the first sensor sensitivity.

2. The analyte sensor system of claim 1, the operations further comprising:
determining that a break-in for the analyte sensor has concluded; and
responsive to determining that the break-in has concluded, determining a second analyte concentration using the sensor signal and a second sensor sensitivity different than the first sensor sensitivity.

3. The analyte sensor system of claim 2, the determining that the break-in for the analyte sensor has concluded comprising determining that a break-in time period has elapsed since the analyte sensor was inserted into the host.

4. The analyte sensor system of claim 3, the break-in time period being about 120 minutes.

5. The analyte sensor system of claim 2, the determining that the break-in for the analyte sensor has concluded comprising determining that a linearity of the sensor signal has reached a linearity threshold.

6. The analyte sensor system of claim 1, the detecting of the property of the sensor signal comprising:
detecting a peak in the sensor signal; and
determining a time-to-peak, the time-to-peak describing a time between when the analyte sensor was inserted into the host and the peak in the sensor signal.

7. The analyte sensor system of claim 1, the operations further comprising:
after accessing the indication that the analyte sensor has been inserted into the host, sampling the sensor signal at a first sample rate; and
after detecting the property of the sensor signal, sampling the sensor signal at a second sample rate, the second sample rate being less than the first sample rate.

8. The analyte sensor system of claim 7, the first sample rate being about one sample per second and the second sample rate being about one sample per 30 seconds.

9. The analyte sensor system of claim 1, the first sensor sensitivity being based on at least one of a time between insertion of the analyte sensor and a rise in the sensor signal or a slope of the sensor signal after insertion.

10. The analyte sensor system of claim 1, the first sensor sensitivity being based at least in part on a first term based on a calibration of the analyte sensor and a second term based on the property of the sensor signal.

11. The analyte sensor system of claim 10, the calibration of the analyte sensor being a factory calibration performed during a manufacturing process of the analyte sensor.

12. The analyte sensor system of claim 10, the first sensor sensitivity being based on an additive combination of the first term and the second term.

13. The analyte sensor system of claim 10, the second term also being based on the calibration of the analyte sensor.

14. The analyte sensor system of claim 1, the first sensor sensitivity being based at least in part on a first term based on a calibration of the analyte sensor, a second term based on the property of the sensor signal, and a third term based on the calibration of the analyte sensor and the property of the sensor signal.

15. The analyte sensor system of claim 14, the first sensor sensitivity being based on an additive combination of the first term, the second term, and the third term.

16. A method of operating an analyte sensor system, the method comprising:
accessing, by sensor electronics of the analyte sensor system, an indication that an analyte sensor has been inserted into a host;
detecting, by the sensor electronics, a property of a sensor signal generated by the analyte sensor after being inserted into the host;
determining, by the sensor electronics, a first sensor sensitivity based at least in part on the property of the sensor signal; and
determining, by the sensor electronics, a first analyte concentration using the sensor signal and the first sensor sensitivity.

17. The method of claim 16, further comprising:
determining, by the sensor electronics, that a break-in for the analyte sensor has concluded; and
responsive to determining that the break-in has concluded, determining, by the sensor electronics, a second analyte concentration using the sensor signal and a second sensor sensitivity different than the first sensor sensitivity.

18. The method of claim 16, the detecting of the property of the sensor signal comprising:
detecting a peak in the sensor signal; and
determining a time-to-peak, the time-to-peak describing a time between when the analyte sensor was inserted into the host and the peak in the sensor signal.

19. The method of claim 16, further comprising:
after accessing the indication that the analyte sensor has been inserted into the host, sampling the sensor signal at a first sample rate; and
after detecting the property of the sensor signal, sampling the sensor signal at a second sample rate, the second sample rate being less than the first sample rate.

20. A non-transitory machine-readable medium comprising instructions thereon that, when executed by at least one processor, cause the processor to perform operations comprising:
accessing an indication that an analyte sensor has been inserted into a host;
detecting a property of a sensor signal generated by the analyte sensor after being inserted into the host;
determining a first sensor sensitivity based at least in part on the property of the sensor signal; and
determining a first analyte concentration using the sensor signal and the first sensor sensitivity.
